SEQUENCE LISTING (3 sequences)

SEQ ID NO: 1 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 2 - protein, 191 aa, Homo sapiens [sequence omitted]

SEQ ID NO: 3 - protein, 217 aa, Homo sapiens [sequence omitted]

(19) United States
(12) Patent Application Publication
(10) Pub. No.: US 2010/0035814 A1
(43) Pub. Date: Feb. 11, 2010

(54) N-Terminal Pegylated Prolactin Receptor Molecules

(75) Inventors: Bernd Peschke, Malov (DK); Leif Christensen, Roskilde (DK); Egon Persson, Ballerup (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/513,578

(22) PCT Filed: Nov. 9, 2007
(86) PCT No.: PCT/EP07/62127

§ 371 Date: Sep. 9, 2009

(30) Foreign Application Priority Data
Nov. 9, 2006 (EP) 06123765.7
Oct. 3, 2007 (JP) 07117814.9

(51) Int. Cl. A61K 38/22; C07K 14/575; C07K 1/107; A61P 35/00

(52) U.S. Cl. 514/12; 530/399; 530/410

(57) ABSTRACT

The invention is concerned with N-terminally pegylated polypeptides capable of binding to the prolactin receptor. Such polypeptides may for instance be N-terminally pegylated antagonists of the prolactin receptor, such as for instance prolactin variants.

### FIELD OF THE INVENTION

The invention relates to novel prolactin receptor antagonist compounds, to pharmaceutical compositions comprising these compounds and to the use of the compounds for the treatment of diseases related to cancer.

### BACKGROUND OF THE INVENTION

According to the World Health Organisation, cancer kills about 7.6 million (or 13%) people worldwide every year. In particular, cancers of the lung, stomach, liver, colon and breast are responsible for over half these deaths.

Recent evidence suggests that there may be a correlation between prolactin expression and inhibition of cancer cells (Wennbo et al. J. of Clin. Invest. 100, 2744-2651 (1997); Liby, et al. Breast Cancer Research and Treatment 79, 241-252 (2003); Clevengeret al. Endocrine Rev. 24, 1-27 (2003)). Prolactin is a single chain polypeptide of 199 amino acids with a molecular weight of about 24,000 Daltons, which is synthesised in the adenohypophysis (anterior pituitary gland), in the breast and in the decidua. Its structure is similar to that of growth hormone (GH) and placental lactogen (PL). The molecule is folded due to the activity of three disulfide bonds.

A simple system is used to describe fragments and analogues of this peptide. For example, G129R-PRL designates an analogue of prolactin formally derived from prolactin by substituting the naturally occurring amino acid residue Glycine (G) in position 129 with Arginine (R). PRL(9-199) and PRL(9-199) designates a fragment formally derived from PRL by removal of the first eight amino acids of the chain.

Six prolactin receptor antagonists are currently known in the literature (Goffin et al. Endocrine Rev. 26, 400-422 (2005)):

(a) G120R/K-hGH, a variant of human growth hormone;

(b) G120R-hPL, a variant of human placental lactogen;

(c) G129R-hPRL a full-length variant of human prolactin;

(d) S179D-hPRL, a full-length variant of human prolactin;

(e) G129R-hPRL (10-199), a truncated variant of human prolactin; and

(f) G129R-hPRL (15-199), a truncated variant of human prolactin.

In vitro experiments with G129R-hPRL and T-47D cells have demonstrated that this antagonist shows an additive effect on the inhibition of proliferation of cells together with tamoxifen (Chen et al. Clin. Cancer Res. 5, 3583 (1999)). The same compound alone has shown in vivo inhibition of tumour growth for T-47D cells (Chen et al. Int. J. Oncology 20, 813-818 (2002)).

However, high levels of these prolactin receptor antagonists are necessary to obtain effects in vivo (Goffin et al. Endocrine Rev. 26, 400-422 (2005)). One way to reduce the levels needed would be to improve the pharmacokinetic parameters, which would result in either the possibility of using lower doses of the prolactin receptor antagonists in question or a more convenient way to sustain the necessary high doses of the prolactin receptor antagonist in question.

Polyethylene glycol (PEG) is a non-toxic polymer with the following structure:

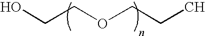

PEGylation is the act of adding a PEG structure to another larger molecule, for example, a therapeutic protein (which is then referred to as PEGylated).

PEGylation has been used for the improvement of pharmacokinetic parameters (Pasut et al. Expert Opin. Ther. Patents 14, 859-894 (2004)). For example, PEGylated prolactin derivatives have been described in U.S. Pat. No. 4,179,337 and US2004/0136952 describes N-terminal PEGylated antagonists of prolactin.

However, PEGylation can affect a protein's binding capabilities. For example, WO2006/024953 describes an N-terminally PEGylated human Growth Hormone, hGH, with a >40 fold loss in binding. Furthermore, Clark et al. (J. Biol. Chem. 271, 21969-21977 (1996)) found a three fold decrease in an in vitro efficacy assay for PEGylated hGH-derivatives when two moieties of 5 kDa PEG were attached randomly to hGH; a 6 to 21 fold decrease in efficacy when three 5 kDa PEG-moieties were attached; and a 44 fold decrease in efficacy when four 5 kDa PEG moieties were attached.

It may therefore be desirable to provide prolactin receptor antagonists linked to PEG having improved pharmacokinetic parameters, but without a significant decrease in the binding to the receptor.

### SUMMARY OF THE INVENTION

The present invention provides a derivatized prolactin molecule comprising a prolactin molecule derivatized in the N-terminus with a group R, which is a bulky group interfering with the binding to the compound to the prolactin receptor.

The present invention provides a compound of formula (I)

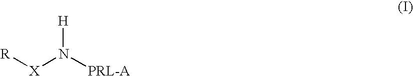

wherein
PRL-A represents a radical of a polypeptide, which polypeptide is capable of binding to the prolactin receptor;
X represents a linker selected from CH₂,

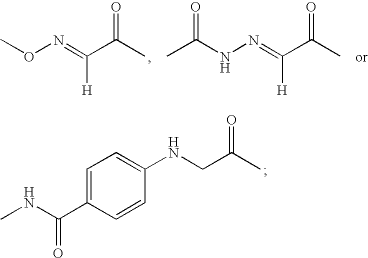

and
R is a radical containing a bulky group interfering with the binding to the compound to the prolactin receptor.

In one embodiment, R is a radical containing a water soluble polymer.

The present invention also provides a compound of formula (Ia):

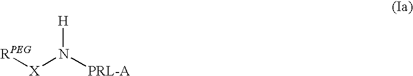

wherein
PRL-A represents a radical of a polypeptide, which polypeptide is capable of binding to the prolactin receptor;
X represents a linker selected from —CH₂—,

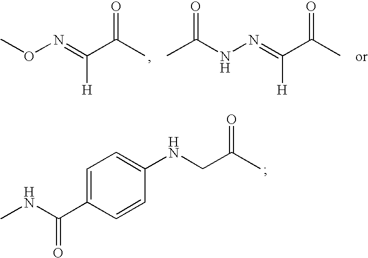

and
R^{PEG} is a polyethylene glycol containing radical.

There is also provided a method of treatment or prophylaxis of cancer, which comprises administration of a compound of formula (I) or (Ia).

There is also provided the use of a compound of formula (I) or (Ia) in the manufacture of a medicament for the treatment or prophylaxis of cancer.

The invention also provides a pharmaceutical composition comprising a compound of formula (I) or (Ia) for use in the treatment or prophylaxis of cancer.

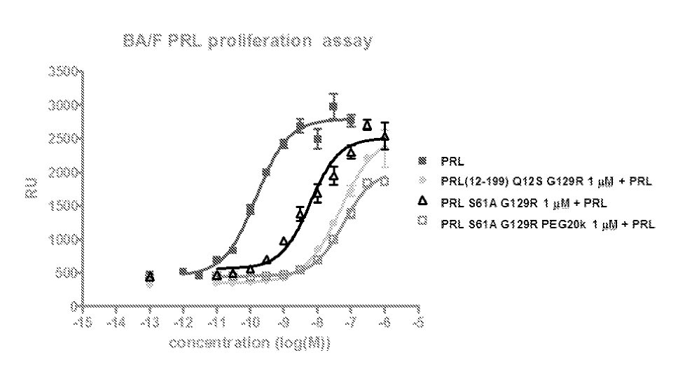

FIG. 1 BaF3 proliferation assay as described in Example 10. Representative results for PRL S61A G129R PEG20k.

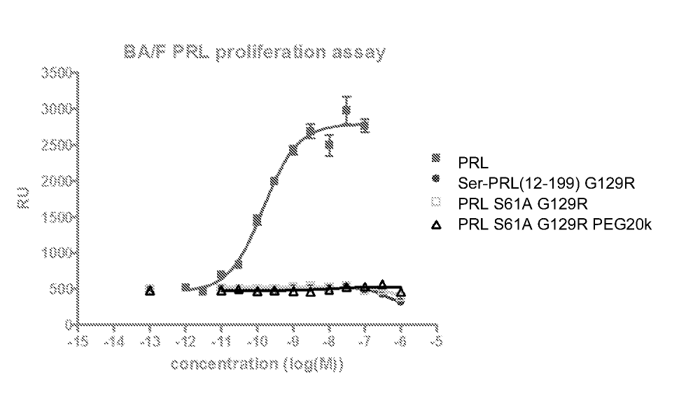

FIG. 2 BaF3 proliferation assay as described in Example 10. Representative results for PRL S61A G129R PEG20k.

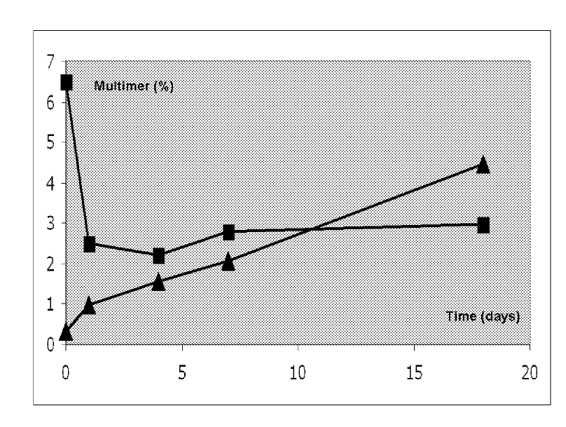

FIG. 3 The time course of multimer formation (determined by SEC analysis) in 215 μM solutions of PRL S61A G129R PEG20k (squares) and its non-pegylated counterpart PRL S61A G129R (triangles) stored at 40° C. in glycyl-glycine buffer, pH 7.5 (150 mM NaCl).

### DETAILED DESCRIPTION OF THE INVENTION

The present invention provides a derivatized prolactin molecule comprising a prolactin molecule derivatized in the N-terminus with a group R, which is a bulky group interfering with the binding to the compound to the prolactin receptor.

The present invention likewise provides a compound of formula (I):

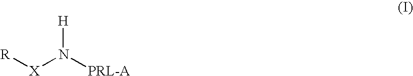

wherein
PRL-A represents a radical of a polypeptide, which polypeptide is capable of binding to the prolactin receptor;
X represents a linker selected from CH₂,

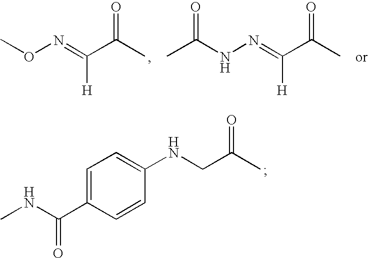

and
R is a radical containing a bulky group interfering with the binding to the compound to the prolactin receptor.

The compounds of the present inventions are useful as antagonists of the PRL receptor.

The radical R may comprise any kind of bulky group, which interferes with the binding to the compound to the prolactin receptor. In one embodiment, R is a polyethylene glycol containing radical.

The present invention provides a compound of formula (Ia):

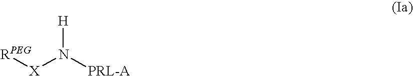

wherein
PRL-A represents a radical of a polypeptide, which polypeptide is capable of binding to the prolactin receptor;
X represents a linker selected from —CH₂—,

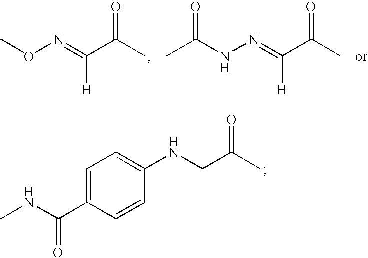

and
R^{PEG} is a polyethylene glycol containing radical.

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, y-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (a-aminoisobutyric acid), Abu (a-aminobutyric acid), Tle (tert-butylglycine), β-alanine, 3-aminomethyl benzoic acid, anthranilic acid.

The production of polypeptides is well known in the art. Polypeptides may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The polypeptides may also be produced by a method which comprises culturing a host cell containing a DNA sequence encoding the polypeptide and capable of expressing the polypeptide in a suitable nutrient medium under conditions permitting the expression of the peptide. For polypeptides comprising non-natural amino acid residues, the recombinant cell should be modified such that the non-natural amino acids are incorporated into the polypeptide, for instance by use of tRNA mutants. The DNA sequence encoding the therapeutic polypeptide may suitably be of genomic or cDNA origin, for instance obtained by preparing a genomic or cDNA library and screening for DNA sequences coding for all or part of the peptide by hybridisation using synthetic oligonucleotide probes in accordance with standard techniques (see, for example, Sambrook, J, Fritsch, E F and Maniatis, T, Molecular Cloning: A Laboratory Manual, Cold Spring Harbor Laboratory Press, New York, 1989). The DNA sequence encoding the polypeptide may also be prepared synthetically by established standard methods, for instance the phosphoamidite method described by Beaucage and Caruthers, Tetrahedron Letters 22, 1859-1869 (1981), or the method described by Matthes et al., EMBO Journal 3, 801-805 (1984). The DNA sequence may also be prepared by polymerase chain reaction using specific primers, for instance as described in U.S. Pat. No. 4,683,202 or Saiki et al., Science 239, 487-491 (1988). The DNA sequence may be inserted into any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, for instance a plasmid. Alternatively, the vector may be one which, when introduced into a host cell, is integrated into the host cell genome and replicated together with the chromosome(s) into which it has been integrated. In one embodiment, the vector is an expression vector in which the DNA sequence encoding the polypeptide is operably linked to additional segments required for transcription of the DNA, such as a promoter, terminator, polyadenylation signals, transcriptional enhancer sequences, and translational enhancer sequences. The vector may also comprise a selectable marker, for instance a gene the product of which complements a defect in the host cell or one which confers resistance to a drug, e.g. ampicillin, kanamycin, tetracyclin, chloramphenicol, neomycin, hygromycin or methotrexate. For large scale manufacture the selectable marker preferably is not antibiotic resistance, e.g. antibiotic resistance genes in the vector are preferably excised when the vector is used for large scale manufacture. Methods for eliminating antibiotic resistance genes from vectors are known in the art, see e.g. U.S. Pat. No. 6,358,705 which is incorporated herein by reference. To direct a parent peptide of the present invention into the secretory pathway of the 30 host cells, a secretory signal sequence (also known as a leader sequence, prepro sequence or pre sequence) may be provided in the recombinant vector. The host cell into which the DNA sequence or the recombinant vector is introduced may be any cell which is capable of producing the present peptide and includes bacteria, yeast, fungi and higher eukaryotic cells. Examples of suitable host cells well known and used in the art are, without limitation, E. coli, Saccharomyces cerevisiae, or mammalian BHK or CHO cell lines. These methods and considerations are well-known to a person skilled in the art.

The term “polyethylene glycol” as used herein means a non-toxic polymer with the following structure:

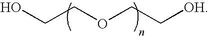

Polypeptides capable of binding to the prolactin receptor may be identified for instance by using the assay described in Example 8 herein. Examples of such polypeptide are prolactin molecules, growth hormone molecules, and placental lactogen molecules.

The term “prolactin molecule” as used herein referring to a polypeptide, which is a prolactin, such as a human prolactin, or an analogue of prolactin, which has the capability of binding to the prolactin receptor. The amino acid sequence of human prolactin is given in SEQ ID No. 1.

The term “growth hormone molecule” as used herein referring to a polypeptide, which is a growth hormone, such as human growth hormone, or an analogue of growth hormone, which has the capability of binding to the prolactin receptor. The amino acid sequence of human growth hormone is given in SEQ ID No. 2.

The term “placental lactogen molecule” as used herein referring to a polypeptide, which is a placental lactogen, such as human placental lactogen, or an analogue of placental lactogen, which has the capability of binding to the prolactin receptor. The amino acid sequence of human placental lactogen is given in SEQ ID No. 3.

The term “analogue” as used herein referring to a polypeptide means a modified peptide wherein one or more amino acid residues of the peptide have been substituted by other amino acid residues and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. All amino acids for which the optical isomer is not stated are to be understood to mean the L-isomer.

The term “prolactin analogue” or “analogue of prolactin” as used herein referring to an analogue of prolactin, which has the capability of binding to the prolactin receptor. In one embodiment, the prolactin analogue has an amino acid sequence having at least 80% identity to SEQ ID No. 1. In one embodiment, the prolactin analogue has an amino acid sequence having at least 85%, such as at least 90%, for instance at least 95%, such as for instance at least 99% identity to SEQ ID No. 1.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci. USA 89, 10915-10919 (1992) for the BLOSUM 62 comparison matrix) is also used by the algorithm.

Preferred parameters for a peptide sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, the prolactin analogue has an amino acid sequence, which sequence is at least 80% similar to SEQ ID No. 1. In one embodiment, the prolactin analogue has an amino acid sequence, which sequence is at least 85%, such as at least 90%, for instance at least 95%, such as for instance at least 99% identity to SEQ ID No. 1.

The term “similarity” is a concept related to identity, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% ((fraction ( 15/20))). Therefore, in cases where there are conservative substitutions, the degree of similarity between two polypeptides will be higher than the percent identity between those two polypeptides.

Conservative modifications of a peptide comprising a given amino acid sequence (and the corresponding modifications to the encoding nucleic acids) will produce peptides having functional and chemical characteristics similar to those of a peptide comprising the given amino acid sequence. In contrast, substantial modifications in the functional and/or chemical characteristics of such peptide as compared to a original peptide may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl. 643, 55-67 (1998); Sasaki et al., Adv. Biophys. 35, 1-24 (1998), which discuss alanine scanning mutagenesis).

Desired amino acid substitutions (whether conservative or non-conservative) may be determined by those skilled in the art at the time such substitutions are desired. For example, amino acid substitutions can be used to identify important residues of the peptides according to the invention, or to increase or decrease the affinity of the peptides described herein for the receptor in addition to the already described mutations.

Naturally occurring residues may be divided into classes based on common side chain properties:

1) hydrophobic: norleucine, Met, Ala, Val, Leu, Iie;

2) neutral hydrophilic: Cys, Ser. Thr, Asn, Gln;

3) acidic: Asp, Glu;

4) basic: H is, Lys, Arg;

5) residues that influence chain orientation: Gly, Pro; and

6) aromatic: Trp, Tyr, Phe.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157, 105-131 (1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those that are within ±1 are particularly preferred, and those within ±0.5 are even more particularly preferred.

It is also understood in the art that the substitution of like amino acids may be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred. One may also identify epitopes from primary amino acid sequences on the basis of hydrophilicity. These regions are also referred to as “epitopic core regions”.

The term “growth hormone analogue” or “analogue of growth hormone” as used herein referring to an analogue of growth hormone, which has the capability of binding to the prolactin receptor. In one embodiment, the growth hormone analogue has an amino acid sequence having at least 80% identity to SEQ ID No. 2. In one embodiment, the growth hormone analogue has an amino acid sequence having at least 85%, such as at least 90%, for instance at least 95%, such as for instance at least 99% identity to SEQ ID No. 2.

In one embodiment, the growth hormone analogue has an amino acid sequence, which sequence is at least 80% similar to SEQ ID No. 2. In one embodiment, the growth hormone analogue has an amino acid sequence, which sequence is at least 85%, such as at least 90%, for instance at least 95%, such as for instance at least 99% identity to SEQ ID No. 2. The term “placental lactogen analogue” or “analogue of placental lactogen” as used herein referring to an analogue of placental lactogen, which has the capability of binding to the prolactin receptor. In one embodiment, the placental lactogen analogue has an amino acid sequence having at least 80% identity to SEQ ID No. 3. In one embodiment, the placental lactogen analogue has an amino acid sequence having at least 85%, such as at least 90%, for instance at least 95%, such as for instance at least 99% identity to SEQ ID No. 3.

In one embodiment, the placental lactogen analogue has an amino acid sequence, which sequence is at least 80% similar to SEQ ID No. 3. In one embodiment, the placental lactogen analogue has an amino acid sequence, which sequence is at least 85%, such as at least 90%, for instance at least 95%, such as for instance at least 99% identity to SEQ ID No. 3.

In one embodiment, PRL-A is a radical of a prolactin receptor antagonist. The term “prolactin receptor antagonist” as used herein means a polypeptide having antagonistic activity at the prolactin receptor. Such prolactin antagonistic activity may be measured by Western blot analysis of the phosphorylation status of STAT5 as set out in Langenheim, J. F. et al, Mol endocrinol. 20(39), 661-674 (2006), for instance as described in Example 8.

In one embodiment, PRL-A is a radical of human prolactin.

In one embodiment, PRL-A is a radical of an analogue of prolactin.

In one embodiment, PRL-A is a radical of an analogue of human prolactin.

In one embodiment PRL-A is a radical of a prolactin analogue in which G129 is exchanged for any other amino acid.

In one embodiment PRL-A is a radical of a prolactin analogue in which S179 is exchanged for any other amino acid.

In one embodiment PRL-A is a radical of any of the following prolactin analogues: G129R-PRL, G129K-PRL, S179D-PRL, S179E-PRL.

In one embodiment PRL-A is a radical of the prolactin analogue G129R-PRL.

In one embodiment PRL-A is a radical of the prolactin analogue: S179D-PRL.

In one embodiment PRL-A is a radical of any of the following prolactin analogues:

### G129R-PRL(9-199);

### G129R-PRL(10-199);

### G129R-PRL(11-199);

### G129R-PRL(12-199);

### G129R-PRL(13-199);

### G129R-PRL(14-199);

### G129R-PRL(15-199);

### C11S G129R-PRL(9-199);

### C11S G129R-PRL(10-199); or

### C11S G129R-PRL(11-199).

In one embodiment PRL-A is a radical of any of the following prolactin analogues:

### G129R-PRL(10-199);

### G129R-PRL(12-199);

### G129R-PRL(14-199); or

### G129R-PRL(15-199).

In one embodiment PRL-A is a radical of:

### C11S G129R-PRL(11-199).

In one embodiment PRL-A is a radical of an hGH-analogue. In a further embodiment the hGH analogues are selected from:

G120R-hGH; and

G120K-hGH.

In one embodiment PRL-A is a radical of an hPL-analogue. In a further embodiment the hPL-analogue is G120-hPL.

In one embodiment, PRL-A carries one or more mutations, which increases the affinity of the molecule to the prolactin receptor as compared to human prolactin (SEQ ID No. 1). In one embodiment, PRL-A has one or more amino acid mutations in the positions corresponding to positions 61, 71 and 73 of SEQ ID No. 1. In one embodiment, the amino acid residue in the position corresponding to position 61 of SEQ ID No. 1 has been substituted with an alanine. In one embodiment, the amino acid residue in the position corresponding to position 71 of SEQ ID No. 1 has been substituted with an alanine. In one embodiment, the amino acid residue in the position corresponding to position 73 of SEQ ID No. 1 has been substituted with an alanine.

In one embodiment, PRL-A has a structure obtainable by the formal removal of an amino-group in the polypeptide capable of binding to the prolactin receptor.

In one embodiment, PRL-A is linked to X via the N-terminus of said PRL-A moiety. In one such embodiment, PRL-A is linked to X via the N-terminal amino acid residue of said PRL-A moiety. In one embodiment, said PRL-A is formally obtained by removal of the N-terminal amino-group.

In one embodiment, R^{PEG} is a PEG-containing radical with an average molecular mass between 1 and 80 kDa, for instance between 5 and 60 kDa, such as between 5 and 40 kDa.

In one embodiment, R^{PEG} is a PEG-containing radical with an average molecular mass selected from: around 1 kDa, around 2 kDa, around 5 kDa, around 10 kDa, around 40 kDA, or around 60 kDa.

In one embodiment, R^{PEG} is a PEG-containing radical, in which at least 85% of the atoms are part of a polymer in which the a repetitive unit is

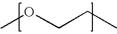

In one embodiment, R^{PEG} has a structure as defined in formula (iia) or (iib)

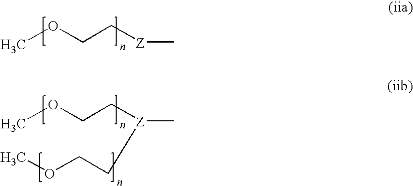

wherein
n is on average between 44 and 1000 and
Z is a linker group, wherein
Z is a bi- or triradical of the formula

R⁵M⁴_{b4}R⁴ₐ₄M³_{b3}R³ₐ₃M²_{b2}R²ₐ₂M¹_{b1}R¹ₐ₁

wherein -R¹-, -R²-, -R³-, and R⁴ independently of each other are biradicals of linear, branched or cyclic C₁₋₁₀alkanes;
—R⁵ is a linear, branched or cyclic C₁₋₁₀alkyl, which is substituted with one or two biradicals of the formula

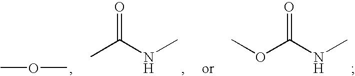

a1, a2, a3, a4, b1, b2, b3, and b4 independently of each other are 0 or 1; and
M¹, M², M³, and M⁴ independently of each other are

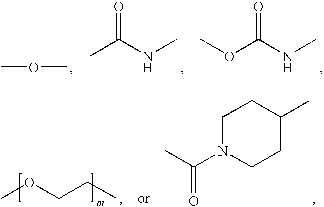

in which m is an integer of from 1 to 115.

The term “C₁₋₁₀alkane” as used herein refers to a straight or branched chain saturated monovalent hydrocarbon molecule having from one to ten carbon atoms, for example C₁₋₈-alkane or C₁₋₆-alkane. Typical C₁₋₈-alkane groups and C₁₋₆-alkane groups include, but are not limited to for instance methan, ethan, n-propan, isopropan, n-butan, sec-butan, isobutan, tert-butan, n-pentan 2-methylbutan, 3-methylbutan, 4-methylpentan, neopentan, n-pentan, n-hexan, 1,2-dimethylpropan, 2,2-dimethylpropan, 1,2,2-trimethylpropan and the like.

The term “C₁₋₁₀alkyl” as used herein refers to a straight or branched chain saturated monovalent hydrocarbon radical having from one to ten carbon atoms, for example C₁₋₈-alkyl or C₁₋₆-alkyl. Typical C₁₋₈-alkyl groups and C₁₋₆-alkyl groups include, but are not limited to for instance methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-pentyl, n-hexyl, 1,2-dimethylpropyl, 2,2-dimethylpropyl, 1,2,2-trimethylpropyl and the like.

In one embodiment, wherein X represents,

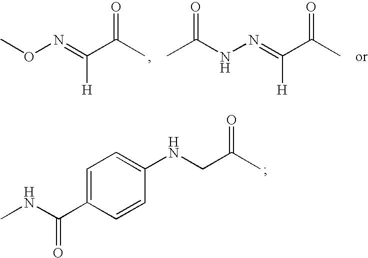

the structure of R^{PEG} is a structure selected from compounds 1-41:

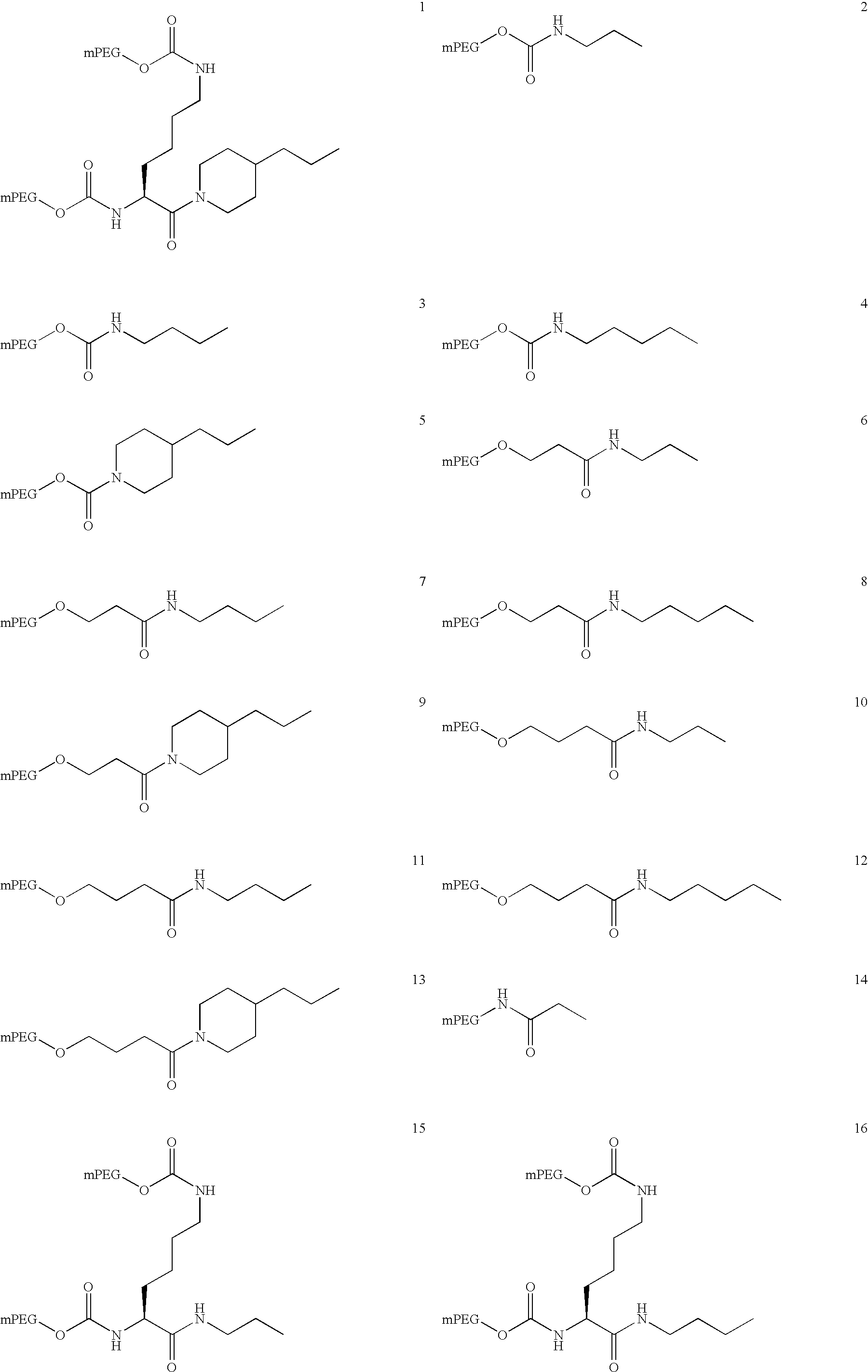

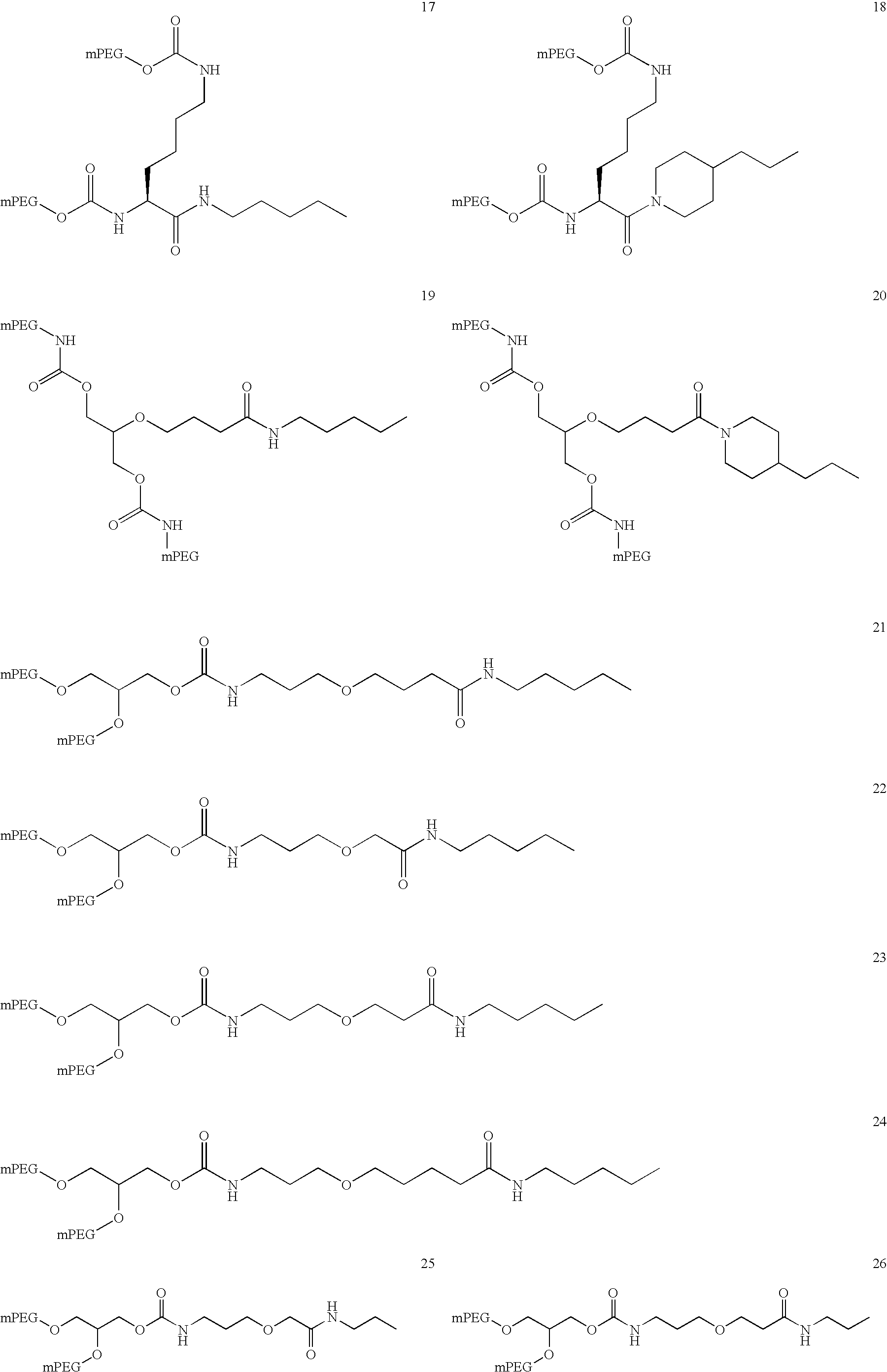

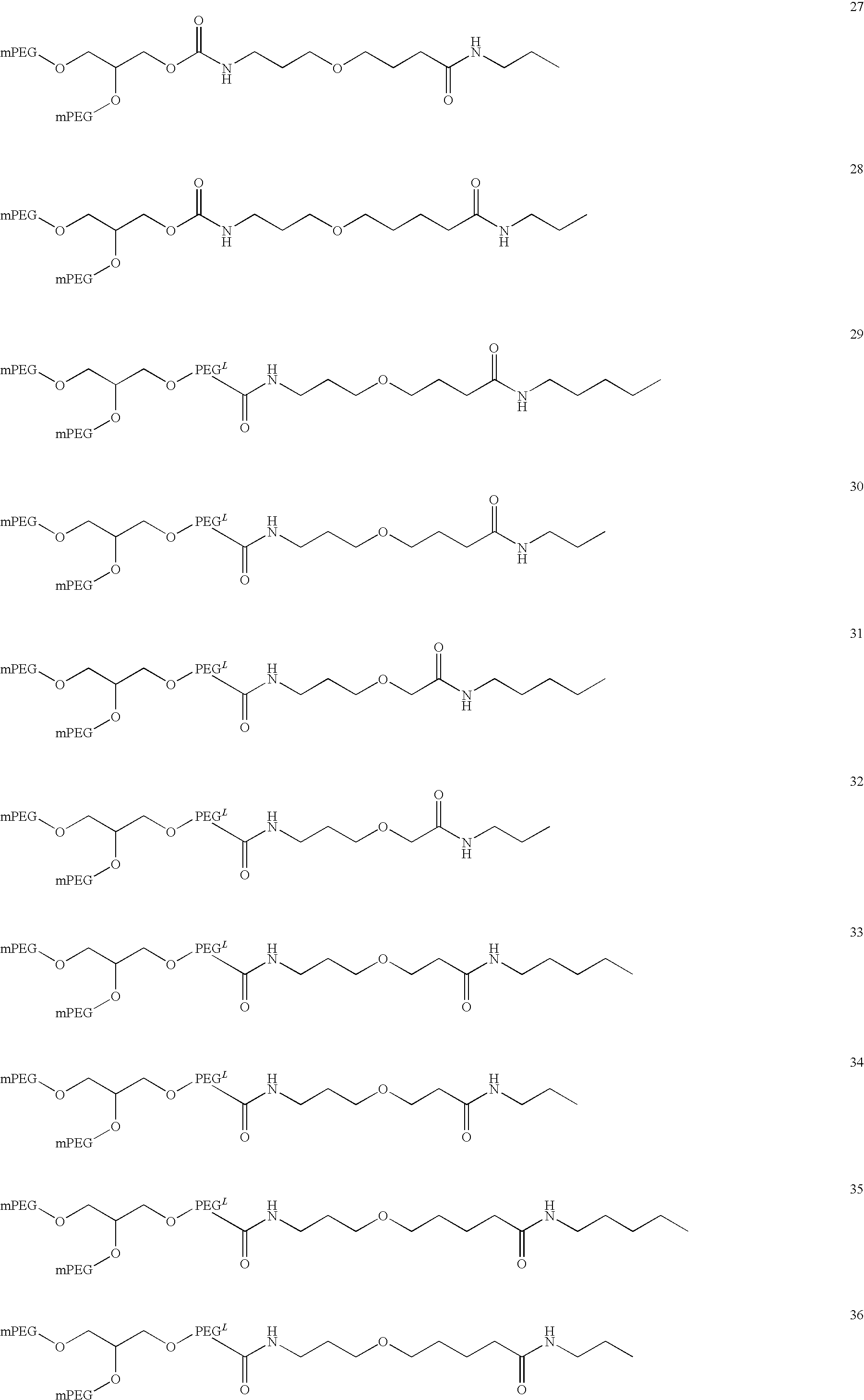

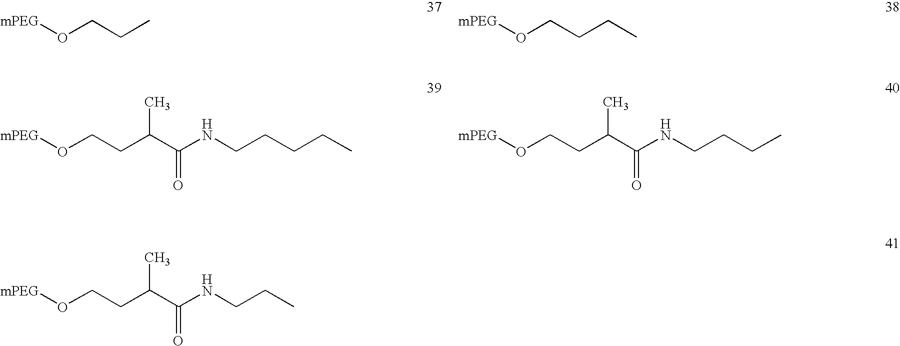

wherein mPEG has a molecular weight of around 10 kDa, around 20 kDa, around 30 kDa or around 40 kDa, and PEG^{L} is a di-radical of a polyethylene glycol-moiety with a molecular weight between 2 kDa and 5 kDa.

In one embodiment, wherein X represents CH₂, the structure of R^{PEG} is a structure selected from compounds 42-49:

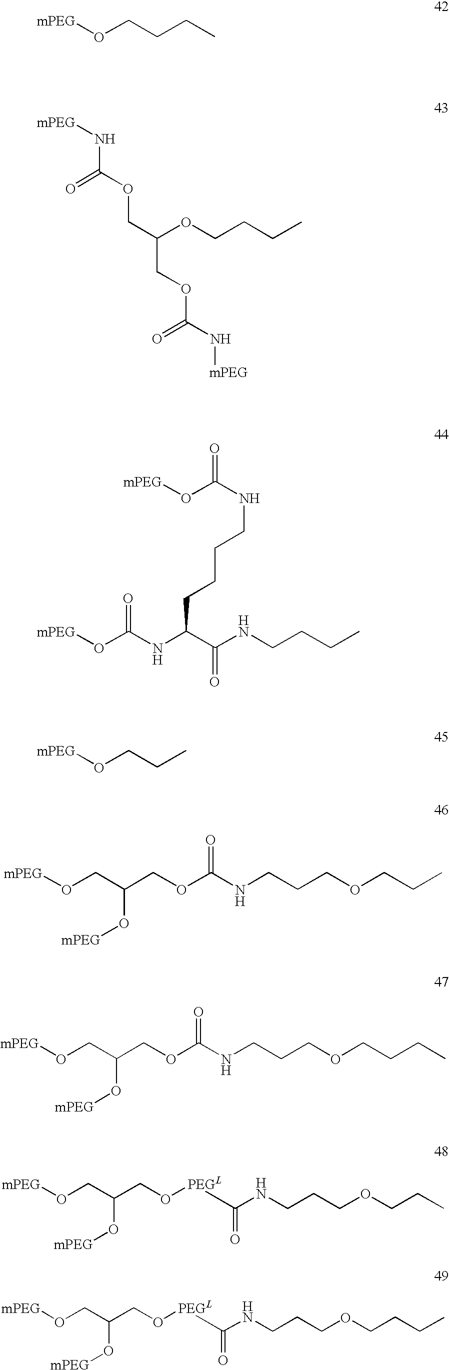

wherein mPEG has a molecular weight of around 10 kDa, around 20 kDa, around 30 kDa or around 40 kDa, and PEG^{L} is a di-radical of a polyethylene glycol-moiety with a molecular weight between 2 kDa and 5 kDa.

In one embodiment, X represents a group of formula:

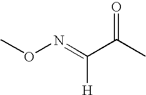

In one embodiment, the compound is selected from a compound of example 2, 3 or 4 (E2-E4).

The invention also provides a process for preparing a compound of formula (Ia) which comprises:

(a) preparing a compound of formula (Ia) wherein X represents:

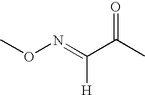

which comprises reaction of formula (II):

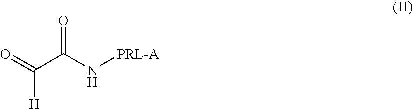

wherein PRL-A is as defined above, with a compound of formula (III):

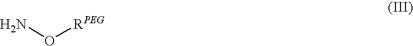

wherein R^{PEG} is as defined above;

or
(b) preparing a compound of formula (Ia) wherein X represents CH₂, which comprises reaction of a compound of formula (IV):

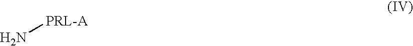

wherein PRL-A is as defined above, with a compound of formula (V):

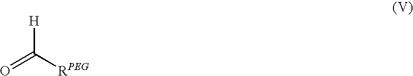

wherein R^{PEG} is as defined above,

or
(c) preparing a compound of formula (Ia), wherein X represents

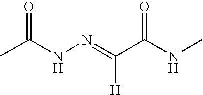

which comprises reaction of a compound of formula (II):

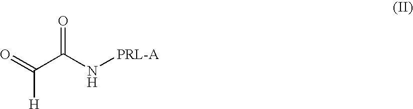

wherein PRL-A is as defined above, with a compound of formula (VII):

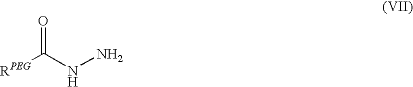

wherein R^{PEG} is as defined above,

or
(d) preparing a compound of formula (Ia), wherein X represents

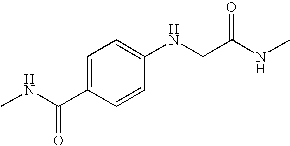

which comprises reaction of a compound of formula (II):

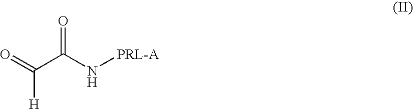

wherein PRL-A is as defined above, with a compound of formula (VIII):

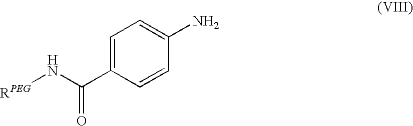

wherein R^{PEG} is as defined above.

Process (a) typically comprises incubation of a compound of formula (II) with a compound of formula (III) at a suitable temperature (e.g. room temperature, 30° C., or 35° C.) in a suitable buffer at a pH of from 2 to 12, particularly from 2 to 7, from 3 to 6, from 3.5 to 5.5, from 4 to 5, from 8 to 12, from 9 to 11, or 10.

Process (b) typically comprises incubation of a compound of formula (IV) and compound of formula (V) with a suitable reduction agent, e.g. sodium cyanoborohydride, sodium borohydride or borane pyridine in an appropriate buffer at a pH of from 2 to 8, suitably from 4 to 8, for instance from 7 to 8.

Typical conditions for process (c) comprise comprises incubation of a compound of formula (II) with a compound of formula (VII) at a suitable temperature (for instance room temperature, 30° C., or 35° C.) in a suitable buffer at a pH of from 2 to 12, particularly from 2 to 7, from 3 to 6, from 3.5 to 5.5, from 4 to 5, from 8 to 12, from 9 to 11, or 10.

Typical conditions for process (d) comprise incubation of a compound of formula (II) and compound of formula (VIII) with a suitable reduction agent, for instance sodium cyanoborohydride, sodium borohydride or borane pyridine in an appropriate buffer at a pH of from 2 to 8.

Compounds of formula (II) may be prepared as described in Scheme 1 below:

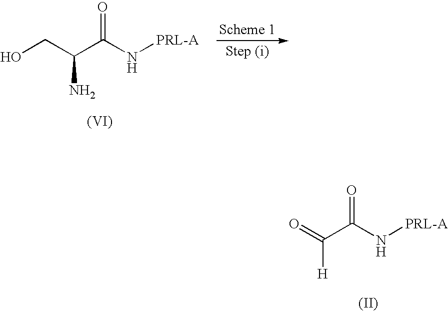

wherein PRL-A is as defined above.

Step (i) may typically be performed by selective oxidization of the N-terminal serine of PRL-A by an appropriate oxidising agent, for instance sodium periodate, in an appropriate buffer, such as triethanolamine,

Compounds of formula (III) and (VI) are either commercially available or may be synthesised in accordance with known procedures.

Compounds of formula (IV) may be produced by peptide synthesis as hereinbefore described.

Compounds of formula (VII) may be prepared from commercially available compound (VIII) by reacting it with hydrazine.

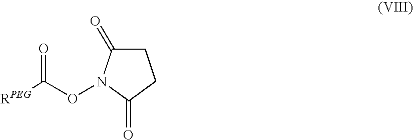

A compound of structure (VIII) may also be prepared for example from a known (for instance Hofmann, Finn, Kiso, J. Am. Chem. Soc 100, 3585-3590 (1978)) compound of structure (IX) with a commercially available PEG reagent (X). The protective group at the amine may be removed by methods known to a person skilled in the art and described for instance in Greene and Wuts “Protective groups in organic synthesis”, 2nd ed. 1991, John Wiley & Sons.

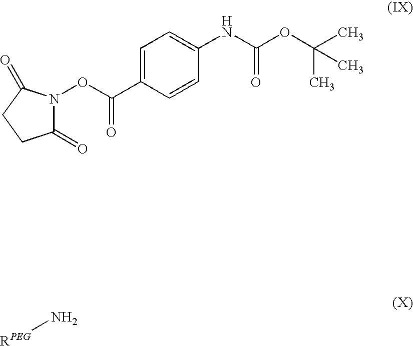

The present invention provides a pharmaceutical composition comprising a compound of formula (Ia) as hereinbefore defined.

The compounds according to the invention are antagonists of the PRL receptor and it is therefore believed that the compounds according to the invention may represent an effective treatment of cancer. Furthermore, the compounds of the present invention have a protracted profile as compared to non-pegylated prolactin.

Wild-type PRL and variants thereof have been shown to form covalently bound multimers involving intermolecular disulfide bonds. With respect to production and formulation the formation of multimers is highly undesirable since such multimeric species can possess unwanted biological or chemical properties. For certain PRL variants that acts as antagonists in their monomeric form, the corresponding dimeric species have been shown to possess agonistic properties (Langenheim, J. F., et al. Molecular endocrinology 20, 661-674 (2006)). Wild-type prolactin contains three disulfide bonds (C4-C11, C58-C174 and C191-C199). Abundant experimental data demonstrate that it is the C4-C11 disulfide bond, situated in the highly flexible N-terminal segment of the hormone, that is primarily involved in the formation of disulfide linked multimers. Accordingly, N-terminal deletion (Δ1-9 to Δ1-14) markedly reduces the propensity of the protein to form oligomers (Bernichtein, S. et al., Molecular and Cellular Endocrinology 208, 11-21 (2003)). Formation of multimers in protein solutions can be measured by size exclusion chromatography (SEC), a chromatographic method that separates molecules according to size. For PRL-variants including the flexible N-terminal segment containing the C4-C11 disulfide bond, N-terminal pegylation was found to reduce the rate of formation of multimeric species, relative to their non-pegylated counterparts (Example 14). N-terminal attachment of a PEG-chain or other bulky moiety may alter the dynamic properties of the intrinsically flexible N-terminal segment and the bulkiness of the PEG-chain make intermolecular disulfide bond formation energetically less favorable. The reduced formation of multimeric species by N-terminal modifications represents an additional advantage of such compounds.

The compounds according to the invention may be used in combination with other therapeutic agents, for example other medicaments claimed to be useful as suitable treatments of cancer, e.g. tamoxifen. When the compounds are used in combination with other therapeutic agents, the compounds may be administered alone or in combination with pharmaceutically acceptable carriers or excipients, in either single or multiple doses, sequentially or simultaneously.

The compounds according to the invention are generally utilised as the free substance or as a pharmaceutically acceptable salt thereof. The term “pharmaceutically acceptable salts” refers to non-toxic salts of the compounds according to the invention, which are generally prepared by reacting the free base with a suitable organic or inorganic acid or by reacting the acid with a suitable organic or inorganic base. When a compound according to the invention contains a free base such salts are prepared in a conventional manner by treating a solution or suspension of the compound with a chemical equivalent of a pharmaceutically acceptable acid. When a compound according to the invention contains a free acid such salts are prepared in a conventional manner by treating a solution or suspension of the antagonist with a chemical equivalent of a pharmaceutically acceptable base. Physiologically acceptable salts of a compound with a hydroxy group include the anion of said compound in combination with a suitable cation such as sodium or ammonium ion. Other salts which are not pharmaceutically acceptable may be useful in the preparation of an prolactin receptor antagonist and these form a further aspect of the present invention.

A pharmaceutical composition according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995. Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining prolactin receptor antagonists and the pharmaceutically acceptable carriers are then readily administered in a variety of dosage forms suitable for the disclosed routes of administration. The formulations may conveniently be presented in unit dosage form by methods known in the art of pharmacy. In addition, some of the prolactin receptor antagonists may form solvates with water or common organic solvents. Such solvates are also encompassed within the scope of the present invention. Thus, in a further aspect, there is provided a pharmaceutical composition comprising a compound according to the invention, or a pharmaceutically acceptable salt, solvate, or prodrug thereof, and one or more pharmaceutically acceptable carriers, excipients, or diluents for use in the treatment of cancer. How to formulate pharmaceutical compostions of polypeptides are well-known in the art and depends for instance the administration route (for instance oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen. The pharmaceutical composition may contain from 0.01% to 100% by weight, for instance from 0.1%-50% by weight, of the compound according to the invention depending on the method of administration. The exact dosage will depend upon the frequency and mode of administration, the sex, age, weight and general condition of the subject treated, the nature and severity of the condition treated and any concomitant diseases to be treated and other factors evident to those skilled in the art.

When compound according to the invention or a pharmaceutically acceptable salt, solvate or prodrug thereof is used in combination with a second therapeutic agent active against the same disease state the dose of each compound may differ from that when the compound is used alone. Appropriate doses will be readily appreciated by those skilled in the art.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law). All headings and sub-headings are used herein for convenience only and should not be construed as limiting the invention in any way. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents. This invention includes all modifications and equivalents of the subject matter recited in the claims appended hereto as permitted by applicable law.

The following is a non-limiting list of embodiments of the present invention.

Embodiment 1: A derivatized prolactin molecule comprising a prolactin molecule derivatized in the N-terminus with a group R, which is a bulky group interfering with the binding to the compound to the prolactin receptor.

Embodiment 2: A derivatized prolactin molecule according to embodiment 1, wherein the prolactin molecule is a prolactin receptor antagonist.

Embodiment 3: A derivatized prolactin molecule according to embodiment 1 or embodiment 2, wherein the prolactin molecule is human prolactin.

Embodiment 4: A derivatized prolactin molecule according to embodiment 1 or embodiment 2, wherein the prolactin molecule is an analogue of prolactin.

Embodiment 5: A derivatized prolactin molecule according to embodiment 4, wherein the prolactin molecule is an analogue of human prolactin.

Embodiment 6: A derivatized prolactin molecule according to any of embodiments 1 to 5, wherein R contains a water soluble polymer.

Embodiment 7: A prolactin molecule according to any of embodiments 1 to 6, wherein R contains a polyethylene glycol group.

Embodiment 8: A compound of formula (I)

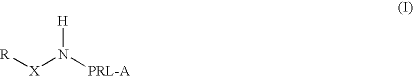

wherein
PRL-A represents a radical of a polypeptide, which polypeptide is capable of binding to the prolactin receptor;
X represents a linker selected from CH₂,

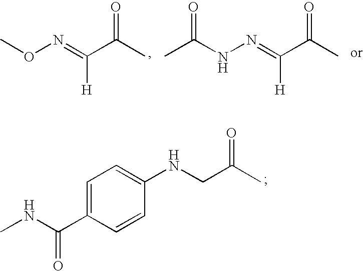

and

R is a radical containing a bulky group interfering with the binding to the compound to the prolactin receptor.

Embodiment 9: A compound according to embodiment 8 of formula (Ia)

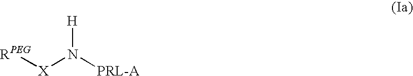

wherein
PRL-A represents a radical of a polypeptide, which polypeptide is capable of binding to the prolactin receptor;
X represents a linker selected from CH₂,

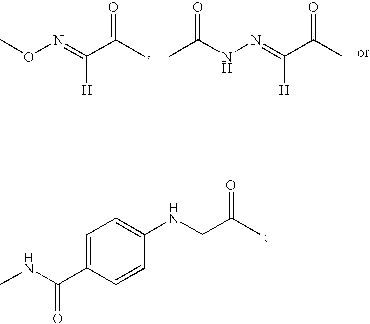

and

R^{PEG} is a polyethylene glycol containing radical.

Embodiment 10: A compound according to embodiment 8 or embodiment 9, wherein PRL-A is a radical of a prolactin receptor antagonist.

Embodiment 11: A compound according to embodiment 8 or embodiment 9, wherein PRL-A is a radical of human prolactin.

Embodiment 12: A compound according to embodiment 8 or embodiment 10, wherein PRL-A is a radical of an analogue of prolactin.

Embodiment 13: A compound according to embodiment 12, wherein PRL-A is a radical of an analogue of human prolactin.

Embodiment 14: A compound according to any of embodiments 8 to 13, wherein PRL-A is linked to X via the N-terminus of said PRL-A moiety.

Embodiment 15: A compound according to embodiment 14, wherein PRL-A is linked to X via the N-terminal amino acid residue of said PRL-A moiety.

Embodiment 16: A compound according to any of embodiments 8 to 15, wherein PRL-A has a structure obtainable by the formal removal of an amino-group in the polypeptide capable of binding to the prolactin receptor.

Embodiment 17: A compound according to any of embodiments 6 to 16, wherein R^{PEG} is a PEG-containing radical with an average molecular mass between 1 and 80 kDa.

Embodiment 18: A compound according to any of embodiments 6 to 17, wherein R^{PEG} is a PEG-containing radical with an average molecular mass selected from: around 1 kDa, around 2 kDa, around 5 kDa, around 10 kDa, around 40 kDA, or around 60 kDa.

Embodiment 19: A compound according to any of embodiments 9 to 18, wherein R^{PEG} is a PEG-containing radical, in which at least 85% of the atoms are part of a polymer in which the a repetitive unit is

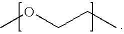

Embodiment 20: A compound according to any of embodiments 9 to 19, wherein R^{PEG} has a structure as defined in formula (iia) or (iib)

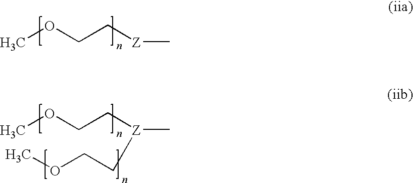

wherein
n is on average between 44 and 1000 and
Z is a linker group, wherein
Z is a bi- or triradical of the formula

R⁵M⁴_{b4}R⁴ₐ₄M³_{b3}R³ₐ₃M²_{b2}R²ₐ₂M¹_{b1}R¹ₐ₁

wherein -R¹-, -R²-, -R³-, and R⁴ independently of each other are biradicals of linear, branched or cyclic C₁₋₁₀alkanes;
-R⁵ is a linear, branched or cyclic C₁₋₁₀alkyl, which is substituted with one or two biradicals of the formula

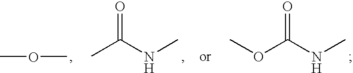

a1, a2, a3, a4, b1, b2, b3, and b4 independently of each other are 0 or 1; and
M¹, M², M³, and M⁴ independently of each other are

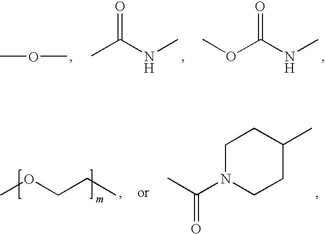

in which m is an integer of from 1 to 115.

Embodiment 21: A compound according to any of embodiments 9 to 20, wherein R^{PEG} has the structure:

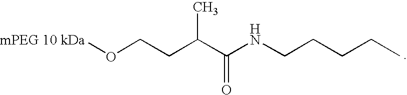

Embodiment 22: A compound according to any of embodiments 9 to 20, wherein R^{PEG} has the structure:

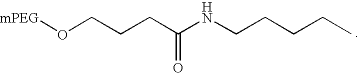

Embodiment 23: A compound according to any of embodiments 9 to 20, wherein R^{PEG} has the structure:

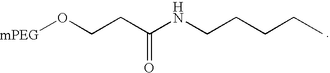

Embodiment 24: A compound according to any of embodiments 8 to 23, wherein X represents a group of formula (i)

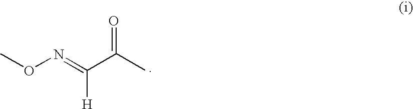

Embodiment 25: A compound as defined in any preceding embodiments, which is:

N^{alpha,13}((4-(2-Methyl-4-(10 kDa mPEGyloxy)butanoylamino)butoxy)imino)acetyl G129R-PRL(13-199) (E2);
N^{alpha,13}((4-(4-(20 kDa mPEGyloxy)butanoylamino)butoxy)imino)acetyl G129R-PRL(13-199) (E3); or
N^{alpha,13}((4-(3-(5 kDa mPEGyloxy)propionylamino)butoxy)imino)acetyl G129R-PRL(13-199) (E4).

Embodiment 26: A process for preparing a compound of formula (Ia) as defined in embodiment 9, wherein X represents a group of formula

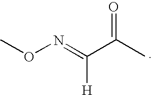

as defined in embodiment 9 which process comprises reaction of a compound of formula (II):

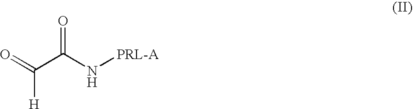

wherein PRL-A is as defined in embodiment 9, with a compound of formula (III):

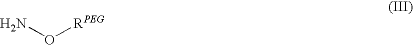

wherein R^{PEG} is as defined in embodiment 9.

Embodiment 27: A process for preparing a compound of formula (Ia) as defined in embodiment 9, wherein X represents CH₂, which process comprises reaction of a compound of formula (IV):

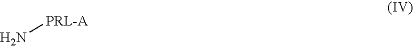

wherein PRL-A is as defined in embodiment 9, with a compound of formula (V):

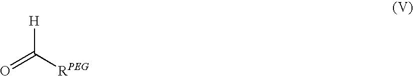

wherein R^{PEG} is as defined in embodiment 9.

Embodiment 28: A process for preparing a compound of formula (Ia) as defined in embodiment 9, wherein X represents

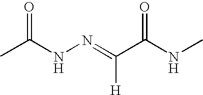

which comprises reaction of a compound of formula (II):

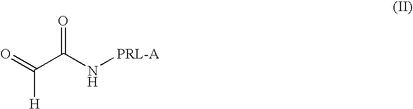

wherein PRL-A is as defined in embodiment 9, with a compound of formula (VII):

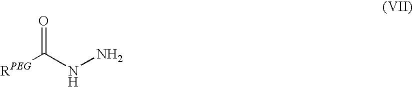

wherein R^{PEG} is as defined in embodiment 9.

Embodiment 29: A process for preparing a compound of formula (Ia) as defined in embodiment 9, wherein X represents

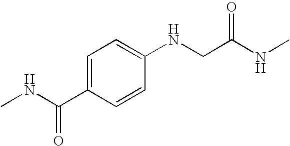

which comprises reaction of a compound of formula (II):

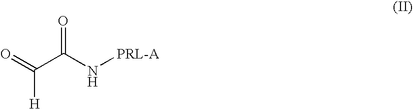

wherein PRL-A is as defined in embodiment 9, with a compound of formula (VIII):

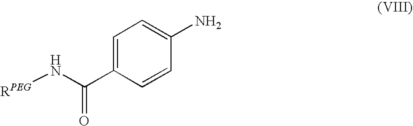

wherein R^{PEG} is as defined in embodiment 9.

Embodiment 30: A compound obtained by a process according to any of embodiments 26 to 29.

Embodiment 31: A compound obtainable by a process according to any of embodiments 26 to 29.

Embodiment 32: A compound according to any of embodiments 1 to 25, embodiment 30 or embodiment 31 for use in therapy.

Embodiment 33: A compound according to any of embodiments 1 to 25, or any of embodiments 30 to 32 for use in the treatment of cancer.

Embodiment 34: A compound according to embodiment 33, wherein said cancer is breast cancer.

Embodiment 35: A pharmaceutical composition comprising a compound according to any of embodiments 1 to 25 or any of embodiments 30 to 34.

Embodiment 36: A pharmaceutical composition comprising a compound according to any of embodiments 1 to 25 or any of embodiments 30 to 34 for use in the treatment or prophylaxis of cancer.

Embodiment 37: A pharmaceutical composition according to embodiment 36, wherein said cancer is breast cancer.

Embodiment 38: A method of treatment or prophylaxis of cancer, which comprises administration of a compound according to any of embodiments 1 to 25 or any of embodiments 30 to 34.

Embodiment 39: A method according to embodiment 38, wherein said cancer is breast cancer.

Embodiment 40: A compound according to any of embodiments 1 to 25 or any of embodiments 30 to 34 in the treatment or prophylaxis of cancer.

Embodiment 41: Use of a compound according to any of embodiments 1 to 25 or any of embodiments 30 to 34 in the treatment or prophylaxis of cancer.

Embodiment 42: Use of a compound according to any of embodiments 1 to 25 or any of embodiments 30 to 34 in the manufacture of a medicament for the treatment or prophylaxis of cancer.

Embodiment 43: Use according to embodiment 41 or embodiment 42, wherein said cancer is breast cancer.

The invention will be further illustrated by the following non-limiting examples.

### EXAMPLES

### Example 1

### General Method (A): Serine Oxidation-Oximation Strategy

A compound 1, which bears a serine residue at its N-terminus, and wherein PRL-A is a radical of a prolactin molecule formally obtained by removal of the N-terminal amino-group, may be prepared biotechnologically by methods known to a person skilled in the art, e.g. in E-coli.

The serine may be oxidized selectively by sodium periodate in an appropriate buffer such as e.g. triethanolamine, furnishing a N-terminal aldehyde. The excess periodate reagent may be quenched with e.g. methionine or 2-hydroxyethyl methyl sulphide.

The buffer of solution containing the protein may be changed to a pH of from 2 to 12, such as from 2 to 7, from 3 to 6, from 3.5 to 5.5, from 4 to 5, from 8 to 12, from 9 to 11, or 10. A PEG-reagent 2, containing an O-substituted hydroxylamine may be added, in which R^{PEG} is any PEG-containing radical. The mixture may be left at a appropriate temperature such as e.g. room temperature, 30° C., or 35° C. The compound may be isolated by methods known to a person skilled in the art e.g. ion exchange chromatography or gel-chromatography.

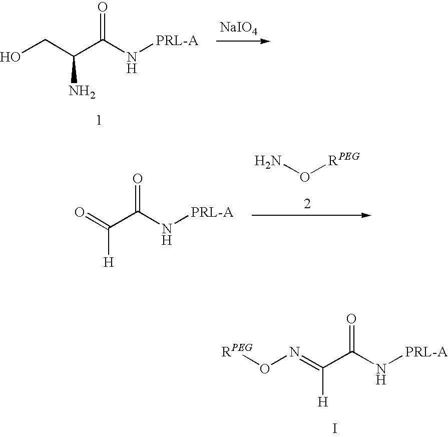

### General Method (B): Reductive Amination Strategy

A compound 3, which PRL-A is a radical of a prolactin molecule formally obtained by removal of the N-terminal amino-group, may be prepared biotechnologically by methods known to a person skilled in the art, e.g. in E. coli.

A PEG-containing aldehyde 4, in which R^{PEG} is any PEG-containing radical, may be added together with a suitable reduction reagent, such as e.g. sodium cyanoborohydride, sodium borohydride or borane-pyridine, in a appropriate buffer such as e.g. an aqueous buffer at a pH of for instance from 2 to 8, from 4 to 8, from 6 to 8, or from 7 to 8. The product may be isolated by methods known to a person skilled in the art e.g. ion exchange chromatography or gel-chromatography.

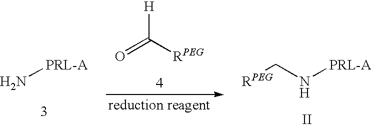

### Example 2

### N^{alpha,13}((4-(2-Methyl-4-(10 kDa mPEGyloxy)butanoylamino)butoxy)imino)-acetyl G129R-PRL(13-199) (E2)

### Step 1: N-(4-(tert-Butoxycarbonylaminoxy)butyl)-2-methyl-4-(10 kDa mPEGyloxy)butanoic amide

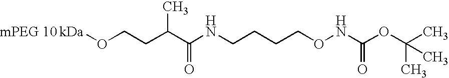

N-(4-Aminobutoxy)carbamic acid tert-butyl ester (204 mg, 1 mmol) was dissolved in dichloromethane (10 ml). The commercially available N-hydroxysuccinimide ester of 2-methyl-4-(10 kDa mPEGyloxy)butanoic acid (1 g, 0.1 mmol) was added. The reaction mixture was stirred for 16 h at room temperature. Ether (90 ml) was added. The formed precipitation was isolated by filtration and dissolved in dichloromethane (10 ml). Ether (90 ml) was added. The formed precipitation was isolated by filtration and dissolved in dichloromethane (6 ml). Ion-exchange material Amberlyst 15 (2 g), which had been washed with dichloromethane (20 ml) and a 10% solution of ethanol in dichloromethane (20 ml), was added. The mixture was gently stirred for 30 min at room temperature. The Amberlyst was filtered off and washed with dichloromethane (10 ml). The combined liquids were concentrated to approx. 2 ml in vacuo. Ether (90 ml) was added. The formed precipitation was isolated by filtration and dried to give 650 mg of N-(4-(tert-butoxycarbonylaminoxy)butyl)-2-methyl-4-(10 kDa mPEGyloxy)-butanoic amide.

### Step 2: N-(4-Aminoxybutyl)-2-methyl-4-(10 kDa mPEGyloxy)butanoic amide

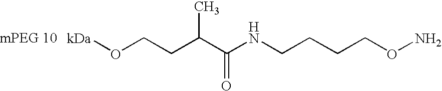

N-(4-(tert-Butoxycarbonylaminoxy)butyl)-2-methyl-4-(10 kDa mPEGyloxy)butanoic amide (650 mg, 0.065 mmol; may be prepared as described in Example 2, Step 1), was dissolved in a mixture of dichloromethane (6 ml) and trifluoroacetic acid (6 ml). The reaction mixture was stirred for 30 min at room temperature. Ether (100 ml) was added. The formed precipitation was isolated by filtration and dissolved in dichloromethane (5 ml). Triethylamine (1 ml) was added. The mixture was stirred for 5 min. Ether (100 ml) was added. The formed precipitation was isolated by filtration, washed with ether (50 ml) and dried in vacuo to give 438 mg of N-(4-aminoxybutyl)-2-methyl-4-(10 kDa mPEGyloxy)butanoic amide.

### Step 3: N^{alpha,13}((4-(2-Methyl-4-(10 kDa mPEGyloxy)butanoylamino)butoxy)imino)acetyl G129R-PRL(13-199)

A solution of N^{alpha,13}seryl G129R-PRL(13-199) (6.25 mg, 284 nmol) in a 50 mM aqueous solution of ammonium hydrogencarbonate (12.3 ml) was concentrated by ultracentrifugation, utilizing an Amicon Ultra filter with a cut-off of 5000 Da. The buffer was changed using the same filter to a solution of triethanolamine (0.004 ml) in water (1.000 ml). A solution 2-hydroxyethyl methylsulfide (0.00223 ml) in water (0.0133 ml) and a solution of sodium periodate (0.45 mg, 2104 nmol) in water (0.00925 ml) were added subsequently. The reaction mixture was gently shaken for 45 min. It was subjected to an ultracentrifugation, utilizing an Amicon Ultra filter with a cut-off of 5000 Da. The buffer was changed using the same filter to a solution of triethanolamine (0.004 ml) in water (1.000 ml). A solution of sucrose (300 mg) in water (0.325 ml) was added. The pH was adjusted to pH 5.05 by addition of a 10% aqueous solution of acetic acid in water. A solution of N-(4-aminoxybutyl)-2-methyl-4-(10 kDa mPEGyloxy)butanoic amide (29 mg, 2865 nmol; may be prepared as described in Example 2, Step 2) in water (0.500 ml), which had been adjusted to pH 4.78 by addition of a 10% aqueous solution of acetic acid in water, was added. The pH of the reaction mixture was found to be 4.98. The reaction mixture was gently shaken at room temperature for 16 h. It was subjected to a gel-chromatography, using a HiPrep26/10 desalting column (GE Healthcare), using a buffer of 25 mM Tris in water, which had been adjusted to pH 8.5 with 1 N hydrochloric acid. The fractions containing the desired product were subjected to an ion-exchange chromatography on a MonoQ 10/100 GL column (GE Healthcare), using a gradient of 0-75% of a buffer consisting of 25 mM Tris and 0.2 M sodium chloride in water, which was adjusted to pH 8.5, in a buffer consisting of 25 mM Tris in water, which was adjusted to pH 8.5, over 30 CV with a flow of 2 ml/min. The fractions, containing the desired product were identified by SDS-electrophoreses. They were pooled and subjected to a gel-chromatography, using a HiPrep26/10 desalting column (GE Healthcare), using a buffer of 50 mM aqueous solution of ammonium hydrogen carbonate to give 2.11 mg of the title compound. The quantification was done at 280 nm, using an extinction coefficient of 9.04. It was characterized by SDS-electrophoreses.

### Example 3

### N^{alpha,13}((4-(4-(20 kDa mPEGyloxy)butanoylamino)butoxy)imino)acetyl G129R-PRL(13-199) (E3)

### Step 1: N-(4-(4-(mPEG20000yloxy)butanoylamino)butoxy)carbamic acid tert-butyl ester

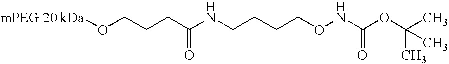

The commercially available N-hydroxysuccinimide ester of mPEG2000yloxybutanoic acid (Nektar “mPEG-SBA”, #2M450P01, 3 g, 0.15 mmol) was dissolved in dichloromethane (25 ml). N-(4-Aminobutoxy)carbamic acid tert-butyl ester (0.12 g, 0.59 mmol) was added. The reaction mixture was shaken at room temperature. Diethyl ether was added until a precipitation was obtained. The precipitation was isolated by filtration. The material was dried in vacuo to yield 2.39 g of N-(4-(4-(mPEG20000yloxy)butanoylamino)butoxy)carbamic acid tert-butyl ester.

### Step 2: N-(4-Aminoxybutyl)-4-(mPEG20000yloxy)butanoylamide

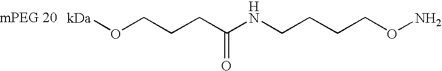

Trifluoroacetic acid (20 ml) was added to a solution of N-(4-(4-(mPEG20000yloxy)butanoylamino)butoxy)carbamic acid tert-butyl ester (2.39 g, 0.12 mmol; may be prepared as described in Example 3, Step 1), in dichloromethane (20 ml). The reaction mixture was shaken for 30 min. Diethyl ether (100 ml) was added. The formed precipitation was isolated by filtration. It was washed with diethyl ether (2×100 ml) and dried in vacuo to give 1.96 g of the trifluoroacetic salt of N-(4-aminoxybutyl)-4-(mPEG20000yloxy)-butanoylamide. A portion of this material (250 mg) was dissolved in water (5 ml) and a 50 mM solution of ammonium hydrogencarbonate (5 ml). It was subjected to gel-chromatography on a HiPrep26/10 desalting column (GE Healthcare) using a 50 mM solution of ammonium hydrogencarbonate as eluent. The fractions containing N-(4-aminoxybutyl)-4-(mPEG20000yloxy)butanoylamide were combined and lyophilized to give the free N-(4-aminoxybutyl)-4-(mPEG20000yloxy)butanoylamide.

### Step 3: N^{alpha,13}((4-(4-(20 kDa mPEGyloxy)butanoylamino)butoxy)imino)acetyl G129R-PRL(13-199)

A solution of N^{alpha,13}seryl G129R-PRL(13-199) (6.25 mg, 284 nmol) in a 50 mM ammonium hydrogencarbonate buffer (12.3 ml) was concentrated by ultracentrifugation using an Amicon Ultra filter with a cut-off of 5000 Da. The buffer was changed to a buffer consisting of 0.004 ml triethanolamine in water (1 ml) by ultracentrifugation using an Amicon Ultra filter with a cut-off of 5000 Da. A solution of 2-hydroxyethyl methyl sulfide (0.00223 ml) in water (0.013 ml) and a solution of sodium periodate (0.45 mg, 2103 nmol) in water (0.00925 ml) were added successively. The mixture was shaken gently for 45 min at room temperature. It was subjected to ultracentrifugation using an Amicon Ultra filter with a cut-off of 5000 Da. The buffer was changed to a buffer consisting of 0.004 ml triethanolamine in water (1 ml) by ultracentrifugation using an Amicon Ultra filter with a cut-off of 5000 Da. A solution of sucrose (300 mg) in water (0.325 ml) was added. The pH was changed to pH 5.06 by addition of a 10% acetic acid in water. A solution of N-(4-aminoxybutyl)-4-(mPEG20000yloxy)butanoylamide (57.3 mg, 2835 nmol; may be prepared as described in Example 3, Step 2), in water, which was adjusted to pH 5.08 by addition of a 10% acetic acid in water, was added. The reaction mixture was gently shaken for 16 h at room temperature. It was subjected to gel chromatography on a HiPrep26/10 desalting column (GE Healthcare) using a 50 mM solution of ammonium hydrogencarbonate as eluent. The protein-containing fractions were pooled and subsequently subjected to ion-exchange chromatography on a MonoQ 10/100 GL column (GE Healthcare), using a gradient of 0-75% of a buffer consisting of 25 mM Tris and 0.2 M sodium chloride in water, which was adjusted to pH 8.5, in a buffer consisting of 25 mM Tris in water, which was adjusted to pH 8.5, over 30 CV with a flow of 2 ml/min. The fractions, containing the desired product were identified by SDS-electrophoreses. They were pooled and subjected to a gel-chromatography, using a HiPrep26/10 desalting column (GE Healthcare), using a buffer of 50 mM aqueous solution of ammonium hydrogen carbonate to give 1.58 mg of the title compound. The quantification was done at 280 nm, using an extinction coefficient of 9.04. It was characterized by SDS-electrophoreses.

### Example 4

### N^{alpha,13}((4-(3-(5 kDa mPEGyloxy)propionylamino)butoxy)imino)acetyl G129R-PRL(13-199) (E4)

### Step 1: 2-(4-(tert-Butoxycarbonylaminoxy)butyl)isoindole-1,3-dione

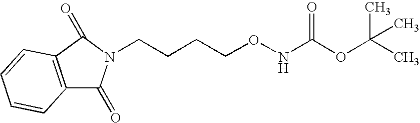

To a mixture of commercially available N-(4-bromobutyl)phthalimide (2.82 g, 10 mmol) and N-Boc-hydroxylamine (2.08 g, 15.6 mmol) was added acetonitrile (2 ml) and successively 1,8-diazabicyclo[5.4.0]undec-7-ene (2.25 ml, 15 mmol). The reaction mixture was stirred at room temperature for 30 min and then at 50° C. for 2 days. It was diluted with a mixture of water (30 ml) and 1 N hydrochloric acid (20 ml). It was extracted with ethyl acetate (2×100 ml). The organic phase was washed with brine (50 ml) and was dried over magnesium sulphate. The crude product was purified by chromatography on silica (60 g), using a gradient of heptane/ethyl acetate 1:0 to 0:1 as eluent to give 2.08 g of 2-(4-(tert-butoxycarbonylaminoxy)butyl)isoindole-1,3-dione.

### Step 2: N-(4-Aminobutoxy)carbamic acid tert-butyl ester

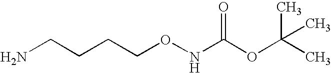

Hydrazine hydrate (1.0 ml, 20 mmol) was added to a solution of 2-(4-(tert-butoxy-carbonylaminoxy)butyl)isoindole-1,3-dione (2.08 g, 6.22 mmol; may be prepared as described in Example 4 Step 1), in ethanol (8.0 ml). The reaction mixture was stirred at 80° C. for 65 h. The solvent was removed in vacuo. The residue was dissolved in toluene (10 ml) and the solvent was removed in vacuo. The residue was suspended in 1 N hydrochloric acid (10 ml). The precipitation was removed by filtration and was washed with water (2 ml). The filtrate and the wash-liquids were combined and made basic with potassium carbonate. The solution was extracted with dichloromethane (4×20 ml). The organic layer was dried over magnesium sulphate. The solvent was removed in vacuo to give 0.39 g of N-(4-amino-butoxy)carbamic acid tert-butyl ester. Potassium carbonate (3 g) was added to the aqueous phase, which was extracted with dichloromethane (3×20 ml). These combined organic layers were dried over magnesium sulphate. The solvent was removed in vacuo to give another 0.39 g of N-(4-aminobutoxy)carbamic acid tert-butyl ester.

### Step 3: N-(4-(3-(5 kDa mPEGyloxy)propionylamino)butoxy)carbamaic acid tert-butyl ester

A solution of N-(4-aminoxybutoxy)carbamaic acid tert-butyl ester (120 mg, 0.584 mmol; may be prepared as described in Example 4, Step 2), was dissolved in dichloromethane (20 ml). 3-(5 kDa mPEGyloxy)propionic acid 2,5-dioxopyrrolidin ester (1.0 g, 0.195 mmol) was added. The reaction mixture was stirred for 16 h. Ether (150 ml) was added. The mixture was stirred for 30 min. The formed precipitation was isolated by filtration and dried in vacuo to give 0.911 g of N-(4-(3-(5 kDa mPEGyloxy)propionylamino)butoxy)carbamaic acid tert-butyl ester.

### Step 4: N-(4-Aminoxybutyl)-3-(5 kDa mPEGyloxy)propionic amide

N-(4-(3-(5 kDa mPEGyloxy)propionylamino)butoxy)carbamaic acid tert-butyl ester (0.911 g, 0.174 mmol; may be prepared as described in Example 4, Step 3), was dissolved in trifluoroacetic acid (8 ml). The mixture was stirred for 30 min at room temperature. Ether (150 ml) was added. The mixture was stirred for 30 min at room temperature. The formed precipitation was isolated by filtration. It was washed with ether (2×50 ml) and dried in vacuo to give 0.80 g of the trifluoroacetate salt of N-(4-aminoxybutyl)-3-(5 kDa mPEGyloxy)-propionic amide. A portion (100 mg) was taken and dissolved in water (5.0 ml). It was subjected to a gel-chromatography on a HiPrep 26/10 desalting column (GE Healthcare), using a 50 mM ammonium hydrogencarbonate buffer. The fractions, containing the compound were lyophilized to give N-(4-aminoxybutyl)-3-(5 kDa mPEGyloxy)propionic amide.

### Step 5: N^{alpha,13}((4-(3-(5 kDa mPEGyloxy)propionylamino)butoxy)imino)acetyl G129R PRL (13-199)

A solution of N^{alpha,13}seryl G129R-PRL(13-199) (8.7 mg, 382 nmol) in a 50 mM aqueous solution of ammonium hydrogencarbonate (18.2 ml) was concentrated by ultracentrifugation, utilizing an Amicon Ultra filter with a cut-off of 5000 Da. The buffer was changed using the same filter to a solution of triethanolamine (0.004 ml) in water (1.000 ml). A solution of 2-hydroxyethyl methyl sulfide (0.003 ml, 0.032 mmol) in water (17.4 ml) was added and a solution of sodium periodate (0.63 mg, 2865 nmol) in water (1.5 ml) were added successively. The reaction mixture was shaken gently for 50 min at room temperature. It was subjected to a ultracentrifugation, utilizing an Amicon Ultra filter with a cut-off of 5000 Da. The buffer was changed using the same filter to a solution of triethanolamine (0.004 ml) in water (1.000 ml). A solution of sucrose (300 mg) in water (0.325 ml) was added. The pH was adjusted to pH 4.83 by addition of a 10% aqueous solution of acetic acid (0.029 ml). A solution of N-(4-aminoxybutyl)-3-(5 kDa mPEGyloxy)propionic amide (20.22 mg, 3820 nmol; may be prepared as described in Example 4, Step 4), in water (0.25 ml), which had been adjusted to pH 5.18 by addition of a 10% aqueous solution of acetic acid, was added. The reaction mixture was gently shaken at room temperature for 16 h. It was subjected to a gel-chromatography, using a HiPrep26/10 desalting column (GE Healthcare), using a buffer of 25 mM Tris in water, which had been adjusted to pH 8.5 with 1 N hydrochloric acid. The fractions containing the desired product were subjected to an ion-exchange chromatography on a MonoQ 10/100 GL column (GE Healthcare), using a gradient of 0-75% of a buffer consisting of 25 mM Tris and 0.2 M sodium chloride in water, which was adjusted to pH 8.5, in a buffer consisting of 25 mM Tris in water, which was adjusted to pH 8.5, over 30 CV with a flow of 2 ml/min. The fractions, containing the desired product were identified by SDS-electrophoreses. They were pooled and subjected to a It was subjected to a gel-chromatography, using a HiPrep26/10 desalting column (GE Healthcare), using a buffer of 50 mM aqueous solution of ammonium hydrogen carbonate to give 2.52 mg of the desired compound. The quantification was done at 280 nm, using an extinction coefficient of 9.04. It was characterized by SDS-electrophoreses.

### Example 5

### N^{alpha1}-(3-(20 kDa-mPEGyl)propyl)PRL S61 G129R

### Preparation of N^{alpha1}-(3-(20 kDa-mPEGyl)propyl)PRL S61 G129R (“PRL S61A G129R PEG20k”)

PRL S61A G129R (10 mg, 433 nmol) was dissolved in a mixture of water (0.200 ml) and ethyldiisopropylamine (0.004 ml). A buffer (0.300 ml) consisting of 25 mM MES, which had been adjusted to pH 6.8 by addition of aqueous sodium hydroxide, was added. A 6 M aqueous solution of sucrose (0.80 ml) was added. The mixture was adjusted to pH 6.77 by addition of a 10% solution of acetic acid in water (0.015 ml). A solution of 3-(20 kDa-mPEGyl)propanal (commercially available at NOF, nr.: Sunbright ME-200AL, 5 mg, 216 nmol) in a buffer (0.300 ml) consisting of 25 mM MES, which had been adjusted to pH 6.8 by addition of aqueous sodium hydroxide, was added. The pH was adjusted to pH 6.68 by addition of a 10% aqueous solution of acetic acid (0.003 ml). A buffer (0.100 ml), consisting of 25 mM MES, which had been adjusted to pH 6.8 by addition of aqueous sodium hydroxide, was added. The mixture was left at room temperature for 15 min. An 1 M aqueous solution of sodium cyanoborohydride (0.0025 ml) was added. The reaction mixture was gently shaken at 21° C. After 1 h, an 1 M aqueous solution of sodium cyanoborohydride (0.0025 ml) was added. Again after 1 h, an 1 M aqueous solution of sodium cyanoborohydride (0.0025 ml) was added. Again after 1 h, an 1 M aqueous solution of sodium cyanoborohydride (0.0025 ml) was added. The reaction mixture was gently shaken at 21° C. for 16 h. It was diluted with a buffer consisting of 25 mM TRIS, which had been adjusted to pH 8.5 by addition of sodium hydroxide, was added, to obtain a total volume of 4 ml. The reaction mixture was filtered. It was subjected to a gel-chromatography, using a HiPrep Desalting 26/10 column and a buffer, consisting of 25 mM TRIS, which had been adjusted to pH 8.5 by addition of sodium hydroxide. The fractions, containing protein were pooled. They were subjected to an anion-exchange chromatography using a MonoQ 10/100 column and a gradient of 0-75% over 30 CV of a buffer consisting of 25 mM TRIS and 0.2 M sodium chloride, which had been adjusted to pH 8.5 by addition of sodium hydroxide, in a buffer consisting of 25 mM TRIS, which had been adjusted to pH 8.5 by addition of sodium hydroxide. The fractions containing the desired protein were pooled according to their purity estimated by SDS-gel electrophoresis. The pool was divided into two parts. Both parts were subjected to a gel-chromatography, using a HiPrep Desalting 26/10 column and a buffer consisting of 50 mM ammonium hydrogencarbonate. All fractions, containing the desired protein were pooled. The desired product was characterized by SDS-gel, which was stained by PEG-sensitive staining as well as silver stain. Both PEG-stain and silver stain methods show one single compound at a MW in accordance for the expectation of N^{alpha1}-(3-(20 kDa-mPEGyl)propyl)PRL S61 G129R. The solution was lyophilized. The residue was redissolved in a buffer consisting of 50 mM ammonium hydrogencarbonate and was filtered to obtain a total volume of 1.7 ml. The concentration was determined by spectrometry at 280 nm using a NanoDrop apparatus, employing an extinction coefficient of 8.97. A protein concentration of 0.27 mg/ml was found. Therefore a yield of 0.857 mg of N^{alpha1}-(3-(20 kDa-mPEGyl)propyl)-PRL S61 G129R was found.

### Example 6

### (4-((4-(20 kDa mPEGyloxy)butanoyl)amino)butyloxyimino)acetylPRL (13-199) S61A G129R

A solution of 2-hydroxyethyl methyl sulphide (0.002 ml, 23000 nmol) in water (0.012 ml) was added to a solution of PRL (12-199) Q12S S61A G129R (6.00 mg, 275 nmol) in a mixture of water (1.00 ml) and triethyanolamine (0.004 ml, 30000 nmol). A solution of sodium periodate (0.46 mg, 2160 nmol) in water (0.012 ml) was added. The reaction mixture was shaken gently at room temperature for 35 min. The buffer was changed by centrifugation in a Biomax centrifugation vial with a cut off of 5000 Da by performing twice a centrifugation at 12500 rpm to a buffer consisting of water (0.200 ml) and triethanolamine (0.0008 ml, 6000 nmol). A solution of sucrose (0.094 mg) in water (0.094 ml) was added. The pH was changed from 9.46 to 5.28 by addition of a 10% aqueous solution of acetic acid (0.014 ml). A solution of N-(4-aminoxybutyl)-4-(mPEG20000yloxy)butanoylamide (55.5 mg, 2750 nmol) in water (0.250 ml) which was adjusted to pH 5.10 by addition of a 10% aqueous solution of acetic acid (0.001 ml) was added. The reaction mixture was gently shaken at room temperature for 16 h. Is was filtrated. It was subjected to a gel chromatography on a HiPrep26/10 desalting column (GE Healthcare) using a 25 mM solution of TRIS, which had been adjusted by addition of hydrochloric acid to pH 8.5. The protein-containing fractions were pooled and subsequently subjected to a ion-exchange chromatography on a MonoQ 10/100 GL column (GE Healthcare), using a gradient of 0-75% of a buffer consisting of 25 mM Tris and 0.2 M sodium chloride in water, which was adjusted to pH 8.5, in a buffer consisting of 25 mM Tris in water, which was adjusted to pH 8.5, over 30 CV with a flow of 2 ml/min. The fractions, containing the desired product were identified by SDS-electrophoreses. They were pooled and subjected to a It was subjected to a gel-chromatography, using a HiPrep26/10 desalting column (GE Healthcare), using a buffer of 50 mM aqueous solution of ammonium hydrogen carbonate to give 1.58 mg of the title compound. The quantification was done at 280 nm, using an extinction coefficient of 9.04. It was characterized by SDS-electrophoreses.

### Example 7

### N^{alpha1}—(N-(4-(4-(N-(2-(2-(2-(20 kDa mPEGylcarbonylamino)ethoxy)ethoxy)-ethyl)carbamoyl)butanoylamino)butoxy)iminoacetyl)-PRL G129R

### Step 1: 4-(N-(4-tert-Butoxycarbonylaminoxybutyl)carbamoyl)butanoic acid

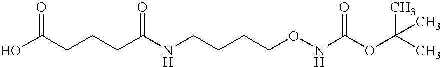

Ethyldiisopropylamine (4.80 ml, 28.0 mmol) was added to a solution of tetrahydropyran-2,6-dione (1.60 g, 14.0 mmol) in a mixture of dichloromethane (15 ml) and N,N-dimethylformamide (5 ml). A solution of N-(4-aminobutoxy)carbamic acid tert-butyl ester (2.86 g, 14.0 mmol) in N,N-dimethylformamide (10 ml) was added. The reaction mixture was stirred for 16 h at room temperature. It was diluted with ethyl acetate (200 ml) and washed with a 10% aqueous solution of sodium hydrogensulphate (100 ml). The aqueous phase was extracted with ethyl acetate (100 ml). The combined organic layers were dried over sodium sulphate. The solvent was removed in vacuo to give 4.18 g of crude 4-(N-(4-tert-butoxy-carbonylaminoxybutyl)carbamoyl)butanoic acid, which was used without further purification in the next step.

MS: m/z=319, required for M+1: 319.

¹H NMR (CDCl₃) δ 1.48 (s, 9H); 1.66 (m, 4H); 1.99 (m, 2H); 2.32 (t, 2H); 2.43 (m, 2H); 3.32 (q, 2H); 3.87 (t, 2H); 6.60 (br, 1H); 7.64 (br, 1H).

### Step 2: 2,5-Dioxopyrrolidinyl 4-(N-(4-tert-butoxycarbonylaminoxybutyl)carbamoyl)butanoic ester

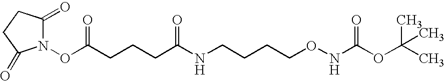

2-Succinimido-1,1,3,3-tetramethyluronium tetrafluoroborate (TSTU, 3.96 g, 13.1 mmol) was added to a solution of triethylamine (1.83 ml, 13.1 mmol) and crude 4-(N-(4-tert-butoxycarbonylaminoxybutyl)carbamoyl)butanoic acid (4.18 g, 13.1 mmol) in N,N-dimethyl-formamide (40 ml). The reaction mixture was stirred for 16 h at room temperature. It was diluted with ethyl acetate (300 ml) and washed with a 10% aqueous solution of sodium hydrogensulphate (200 ml). The aqueous phase was extracted with ethyl acetate (100 ml). The combined organic layers were washed with a mixture of brine (100 ml) and water (100 ml) and dried over sodium sulphate. The solvent was removed in vacuo. A part of the crude product was purified by flash chromatography on silica (40 g), using ethyl acetate as eluent to give 522 mg of 2,5-dioxopyrrolidinyl 4-(N-(4-tert-butoxycarbonylaminoxybutyl)carbamoyl)-butanoic ester.

MS: m/z=416, required for M+1: 416.

¹H NMR (CDCl₃) δ 1.48 (s, 9H); 1.65 (m, 4H); 2.10 (quintet, 2H); 2.31 (t, 2H); 2.68 (t, 2H); 2.86 (m, 4H); 3.30 (q, 2H); 3.86 (t, 2H); 6.37 (br, 1H); 7.31 (br, 1H).

### Step 3: N-(4-(4-(N-(2-(2-(2-(20 kDa mPEGylcarbonylamino)ethoxy)ethoxy)ethyl)-carbamoyl)butanoylamino)butoxy)carbamic acid tert-butyl ester

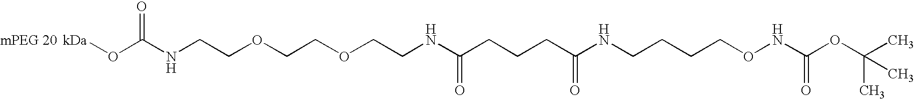

A solution of 2,5-dioxopyrrolidinyl 4-(N-(4-tert-butoxycarbonylaminoxybutyl)-carbamoyl)butanoic ester (0.247 g, 0.594 mmol) in dichloromethane (25 ml) was added to a solution of 2-(2-(2-(20 kDa mPEGylcarbonylamino)ethoxy)ethoxy)ethylamine (commercially available at Nektar, 3.0 g, 0.15 mmol) and triethylamine (0.12 ml, 0.891 mmol) in dichloromethane. The reaction mixture was stirred for 16 h at room temperature. Ether (500 ml) was added. The formed precipitation was left for 30 min and isolated by filtration through a P2-glas filter. It was washed with ether (100 ml) and dried in vacuo to give 2.72 g of N-(4-(4-(N-(2-(2-(2-(20 kDa mPEGylcarbonylamino)ethoxy)ethoxy)ethyl)carbamoyl)butanoyl-amino)butoxy)carbamic acid tert-butyl ester.

### Step 4: N-(4-Aminoxybutyl)-4-(N-(2-(2-(2-(20 kDa mPEGylcarbonylamino)ethoxy)-ethoxy)ethyl)carbamoyl)butanoic amide

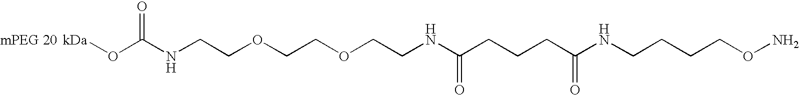

N-(4-(4-(N-(2-(2-(2-(20 kDa mPEGylcarbonylamino)ethoxy)ethoxy)ethyl)carbamoyl)-butanoylamino)butoxy)carbamic acid tert-butyl ester (2.72 g, 0.133 mmol) was dissolved in dichloromethane (15 ml). Trifluoroacetic acid (15 ml) was added. The reaction mixture was stirred for 1.3 h at room temperature. Ether (500 ml) was added. The formed precipitation was left for 15 min and subsequently isolated by filtration through a P3-glas filter. It was washed with ether (2×50 ml) and dried in vacuo. The material was subjected in two parts to a gel-chromatography on a G25 desalting column with a flow of 7 ml/min. The fractions containing the desired material were lyophilized to give 206 mg of N-(4-aminoxybutyl)-4-(N-(2-(2-(2-(20 kDa mPEGylcarbonylamino)ethoxy)ethoxy)ethyl)carbamoyl)butanoic amide.

### Step 5

A solution of 2-hydroxyethyl methyl sulfide (0.00096 ml, 11050 nmol) in water (0.020 ml) and a solution of sodium periodate (0.000208 mg, 975 nmol) in water (0.015 ml) were subsequently added to a solution of N^{alpha1}-seryl-PRL G129R (3 mg, 130 nmol) in a solution of triethanolamine (0.00119 ml, 8970 nmol) in water (1 ml). The reaction mixture was gently shaken at room temperature for 15 min. It was transferred to an Amicon Ultra centrifugal filter device (Millipore) with a cut off of 5 kDa and was concentrated at a speed of 4000 rpm for 5 min. A solution of triethanolamine (0.00595 ml, 44850 nmol) in water (5 ml) was added. It was concentrated at a speed of 4000 rpm for 10 min. A solution of triethanolamine (0.00595 ml, 44850 nmol) in water (5 ml) was added. It was concentrated at a speed of 4000 rpm for 10 min. The reaction mixture was transferred into an Eppendorf vial. A solution of triethanolamine (0.000357 ml, 2690 nmol) was added to obtain a total volume of 1.60 ml. A solution of sucrose (26.6 mg) in water (0.133 ml) was added to the solution. The pH was adjusted to pH 5.3 by addition of a 10% aqueous acetic acid (0.010 ml). N-(4-aminoxybutyl)-4-(N-(2-(2-(2-(20 kDa mPEGylcarbonylamino)ethoxy)ethoxy)ethyl)carbamoyl)butanoic amide (27 mg, 1300 nmol) was dissolved in water (0.150 ml). The pH of this solution was found to be 5.3. This solution was added to the solution containing the protein. It was gently shaken at room temperature for 16 h. The solution was frozen until purification.

The sample was thawed and transferred into an Amicon Ultra centrifugal filter device (Millipore) with a cut off of 5 kD. A buffer consisting of 25 mM TRIS, which was adjusted to pH 8.5 with hydrochloric acid (3 ml) was added. The solution was concentrated at a speed of 4000 rpm for 5 min. A A buffer consisting of 25 mM TRIS, which was adjusted to pH 8.5 with hydrochloric acid (3 ml) was added. The solution was concentrated at a speed of 4000 rpm for 5 min. A buffer consisting of 25 mM TRIS, which was adjusted to pH 8.5 with hydrochloric acid (3 ml) was added. The solution was concentrated at a speed of 4000 rpm for 5 min. The solution was subjected to an ion-exchange chromatography on a MonoQ 10/100 GL column, using a gradient of 0-75% of a buffer consisting of 25 mM TRIS, 0.2 M sodium chloride, which had been adjusted to pH 8.5 in a buffer of 25 mM TRIS, which had been adjusted to pH 8.5, over 30 column volumes at a flow of 4 ml/min (0.5 ml/min during application onto the column and wash-out) to elute the product. The fractions containing the desired product were combined and were subjected to a G25-gel HighPrep Desalting column using a 50 mM solution of ammonium hydrogencarbonate as eluent. The solution was concentrated in an Amicon Ultra centrifugal filter device (Millipore) with a cut off of 5 kDa at a speed of 4000 rpm for 10 min. The yield was determined by photometric methods at 280 nm on a Nanodrop ND 1000 instrument, using an absorption coefficient of 8.97 for the protein-part of the compound. The total yield was found go be 0.207 mg. It was characterized by SDS-gel electrophoresis, which was in accordance with the expectation.

### Example 8

### Assay Methodology

The soluble form of the PRL receptor (25 μg/ml in 10 mM sodium acetate, pH 3.0) was injected into a Biacore 3000 instrument at a flow rate of 5 μl/min and coupled to a CM5 sensor chip by amine coupling chemistry. Prolactin molecules (500 nM in buffer; 20 mM Hepes, pH 7.4, containing 0.1 M NaCl, 2 mM CaCl₂ and 0.005% P20) were then injected over the immobilized receptor for 5 minutes at the same flow rate, followed by a 10-min dissociation period during which buffer was injected, to assess receptor binding affinity. Data evaluation was performed in BiaEvaluation 4.1. Regeneration was accomplished with 4.5 M MgCl₂ between runs.

### Example 9

### Binding to the Prolactin Receptor of N-Terminally Pegylated Prolactin Antagonists

Using the assay as described in Example 8, the binding of N-terminally pegylated versions of the prolactin antagonist PRL(12-199) Q12S G129R to the prolactin receptor was determined. The results are shown in Table 1.

TABLE 1

| Name | Kd (nM) |
|---|---|
| PRL wildtype | 32 |
| PRL(11-199) C11S G129R prepared as in | 55 |
| Example 2 | |
| PRL(11-199) C11S G129R prepared as in | 70 |
| Example 3 | |
| PRL(11-199) C11S G129R prepared as in | 42 |
| Example 4 | |
| PRL Q12S S61A G129R prepared as in Example 5 | 71 |

These prolactin antagonists may thus be N-terminally pegylated without any detrimental loss in binding to the prolactin receptor.

### Example 10

### BaF3 Proliferation Assay

BaF/3 cells stably transfected with hPRLR were maintained in the full growth RPMI1640 medium supplemented with 2 mM L-glutamine, 10% FCS and 10 ng/ml wtPRL. Cell were splitted approx. every third day. Prolactin was added upon splitting. Before running the assay, the cells were grown in the medium omitting PRL for 24 hours. The cells were resuspended in fresh medium to 5×10⁵ cells/ml. 100 μl of the cells were fed into wells of a 96-well plate, 50 μl of agonist or wtPRL(1 nM)/antagonists at different concentrations were added to the cells, and the cells were incubated for 68 hours. 50 μl of AlamarBlue (medium: AlamarBlue reagent=7:1) was added to each well, and the cells were then incubated for additional 4 hours. The fluorescence was measured on a BMG LABTECH microplate reader using Ex 544 nm; Em 590 nm. Representative results for PRL S61A G129R PEG20k (as prepared in Example 5 are shown in FIGS. 1 and 2.

The antagonistic effect of Ser PRL S33A Q73L G129R K190R and its pegylated derivative Ser PRL S33A Q73L G129R K190R PEG20k was also measured. The results are shown in Table 2.

TABLE 2

| Compound | EC50 (nm) |
|---|---|
| PRL | 0.15 ± 0.08 |
| PRL in the presence of 1 μM PRL(12-199) Q12S G129R | 40 ± 18 |
| PRL in the presence of 1 μM Ser PRL S33A Q73L | 531 ± 286 |
| G129R K190R | |
| PRL in the presence of 1 μM Ser PRL S33A Q73L | 19 ± 6 |
| G129R K190R PEG20k | |

Data is presented as mean ± SD of n independent experiments performed in triplicate

The antagonistic effect of PRL S61A G129R and its pegylated analogue PRL S61A G129R PEG20k was also measured. The results are shown in Table 3.

TABLE 3

| Compound | EC50 (nM) |
|---|---|
| PRL | 0.15 ± 0.08 |
| PRL in the presence of 1 μM PRL S61A G129R | 116 ± 68 |
| PRL in the presence of 1 μM PRL S61A G129R PEG20k | 4.6 ± 3.8 |

Data is presented as mean ± SD of n independent experiments performed in triplicate

### Example 11

### N^{alpha1}-(3-(20 kDa-mPEGyl)propyl) Ser PRL S33A Q73L G129R K190R

### Preparation of N^{alpha1}-(3-(20 kDa-mPEGyl)propyl) Ser PRL S33A Q73L G129R K190R (“PRL Ser PRL S33A Q73L G129R K190R PEG20k”)

Ser PRL S33A Q73L G129R K190R (480 mg, 20.8 μmol) was dissolved in 33 ml 100 mM MOPS (3-morpholinopropansulfonic acid) buffer which had been adjusted to pH 6.8 by addition of aqueous sodium hydroxide. A solution of 3-(20 kDa-mPEGyl)propanal (commercially available at NOF, nr.: Sunbright ME-200AL, 300 mg, 13 μmol) in a 100 mM MOPS buffer adjusted to pH 6.8 by addition of aqueous sodium hydroxide, was added. The mixture was left at room temperature for 5 min. An 1 M aqueous solution of sodium cyanoborohydride (480 μL) was added. The reaction mixture was gently shaken at 20° C. Again after 1 h, an 1 M aqueous solution of sodium cyanoborohydride (480 μL) was added. The reaction mixture was gently shaken at 20° C. for 3 h. The protein was taken into a 20 mM triethanolamine buffer (70 ml) pH 8.5 using PD-10 columns (Sephadex®G-25M, Amersham pharmacia, 17-0851-01). The protein was subjected to a anion exchange chromatography using a Q sepharose HP (GE lifescience) column (Buffer A: 20 mM triethanolamine, pH 8.5, Buffer B: 20 mM triethanolamine, 0.2 M NaCl, pH 8.5, equilibration 5 cv, Load 175 ml, wash 10 cv, Elution gradient 0-50% Buffer B in 30 cv). The fractions containing the desired protein were pooled according to their purity estimated by SDS-gel electrophoresis. The desired product was characterized by both by HPLC, using a Phenomenex C4 Jupiter (cat#00G-4167-E0) column (Buffer A: 0.1% TFA aqueous, Buffer B: 0.07% TFA in CH3CN, 1 ml/min, 42° C., linear gradient 40-90% Buffer B. 20 min) and SDS-gel, which was stained by PEG-sensitive staining as well as silver stain. Both PEG-stain and silver stain methods show one single compound at a MW in accordance for the expectation of N^{alpha1}-(3-(20 kDa-mPEGyl)propyl) Ser PRL S33A Q73L G129R K190R. The solution was lyophilized. The residue was redissolved in a buffer consisting of 50 mM ammonium hydrogencarbonate and was filtered. Yield: 106 mg of purified protein was obtained (>90% purity by HPLC)

### Example 12

### Pharmacokinetics of PEGylated Prolactin Receptor Antagonist after Intravenous and Subcutaneous Administration in Nude Mice

The study was performed in 18 female nude NMRI mice from Charles River, Sulzfeld, Germany. The body weight was in the range of 19-28 g. The mice were allowed free access to feed and water. PEGylated prolactin receptor antagonist was used for dosing. The test substance was diluted in a PBS buffer (150 mM NaCl, 10 mM PO₄, 3 mM KCl), pH 7.5 and stored at 4° C. until use. The mice were divided into two groups with 9 mice pr group. One group received a single intravenous (iv) administration in the tail vein; the other group received a single subcutaneous (sc) bolus in the right flank. The mice receiving the sc dose were anaesthetized by Isofluran/O₂/N₂O (Isofluran: Forene inhalation fluid, no 506949, Abbott Scandinavia AB, Solna, Sweden) before injection. All the animals were dosed 200 μg PEGylated prolactin receptor antagonist per mouse corresponding to 4.4 nmol PEGylated prolactin receptor antagonist per mouse. Samples were taken in sparse sampling schedule (3 mice per time point) at predose, 0.25, 0.5, 1, 2, 4, 7, 18, 24 hours post administration. Before blood sampling the mice were anaesthetized by Isofluran/O₂/N₂O. Approximately 50 μl blood (˜4 droplets) was sampled in from the eye using a 10 μl capillary glass tube. Blood samples were collected in Eppendorf tubes and stored for at least 1 hour at room temperature. Thereafter the blood samples were centrifuged at 4000 RPM for 5 minutes and the serum supernatants (approximately 25 μl) were collected in Micronic tubes, placed in racks and stored at −80° C. After the third blood sample, the mice were killed by cervical dislocation. The serum concentration of PEGylated prolactin receptor antagonist was determined using an enzyme immunoassay kit (DSL kit, product number DSL-10-4500). The assay calibration curve was constructed with defined quantities of the antagonist spiked into phosphate-buffered saline solution, pH 7.4. Results from the enzyme immunoanalysis were subjected to non-compartmental pharmacokinetic analysis using the PC based software WinNonlin (Pharsight Corporation). Bioavailability (F) was calculated as:

$$F = \frac{{AUC}_{s . c .}}{{AUC}_{i . v .}} \times \frac{{Dose}_{i . v .}}{{Dose}_{s . c .}}$$

### Example 13

### Intravenous and Subcutaneous Administration of PEGylated Prolactin Receptor Antagonist to Nude Mice

Using the assay as described in Example 12, the pharmacokinetics of N-terminally pegylated versions of the prolactin antagonist PRL(12-199) Q12S G129R were determined.

Pharmacokinetic parameters for iv administration as obtained by NCA

| | Actual | | | | | | |
|---|---|---|---|---|---|---|---|
| | dose | | AUC_{0-∞} | | | | |
| Com- | (μg/ | Cₘₐₓ | (h * | AUCₑₓₜᵣₐₚ | HL | Cl | V_{z} |
| pound | mouse) | (ng/ml) | ng/l) | (%) | (h) | (ml/h) | (ml) |
| PEGylated | 230 | 253058 | 997444 | 0.53 | 3.1 | 0.23 | 1.04 |
| prolactin | | | | | | | |
| receptor | | | | | | | |
| antagonist | | | | | | | |
| as pre- | | | | | | | |
| parred in | | | | | | | |
| example 5 | | | | | | | |

Pharmacokinetic parameters for sc administration as obtained by NCA

| | Actual | | | | | | | | |
|---|---|---|---|---|---|---|---|---|---|
| | dose | | | | | | | | |
| | (μg/ | Tₘₐₓ | Cₘₐₓ | AUC_{0-∞} | AUCₑₓₜᵣₐₚ | HL | Cl/F | V_{z}/F | F |
| Compound | mouse) | (h) | (ng/ml) | (h * ng/l) | (%) | (h) | (ml/h) | (ml) | (%) |
| PEGylated | 230 | 4 | 59387 | 746750 | 18 | 6.5 | 0.31 | 2.88 | 75 |
| prolactin | | | | | | | | | |
| receptor | | | | | | | | | |
| antagonist | | | | | | | | | |
| as pre- | | | | | | | | | |
| parred in | | | | | | | | | |
| example 5 | | | | | | | | | |

### Example 14

### Formation of Multimeric Species Upon Storage

PRL S61A G129R PEG20k and its non-pegylated counterpart PRL S61A G129R at equal molar concentration (215 uM) were kept at 40° C. in glycyl-glycine buffer, pH 7.5 (150 mM NaCl). Immediately after preparation and then again after 1, 4, 7 and 18 days the samples (10 μl aliquots) were analyzed by size exclusion chromatography (SEC): BioSep-SEC-S3000 (300×7.6 mm) eluted with PBS-buffer (10 mM phosphate buffer, 150 mM NaCl, 3 mM KCl, pH 7.5) at a flow rate of 0.8 ml/min, UV detection at 215 and 280 nm). Multimer contents (%) were calculated from the chromatogram detected at 280 nm. The time course of multimer formation for PRL S61A G129R PEG20k and PRL S61A G129R are depicted in FIG. 3. The initial multimer content in S61A G129R PEG20k is relatively high (6.5%), but is markedly reduced at day 1 (2.5%) and then levels out at ˜3% after day 7. In contrast to this profile, the multimer content of the non-pegylated analogue PRL S61A G129R steadily increases from day 0.

1. A derivatized prolactin molecule comprising a prolactin molecule derivatized in the N-terminus with a group R, which is a bulky group interfering with the binding to the compound to the prolactin receptor.

2. A prolactin molecule according to claim 1, wherein R contains a polyethylene glycol group.

3. A compound of formula (I) 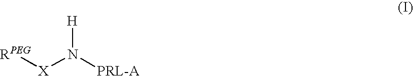
wherein
PRL-A represents a radical of a polypeptide, which polypeptide is capable of binding to the prolactin receptor;
X represents a linker selected from CH₂, 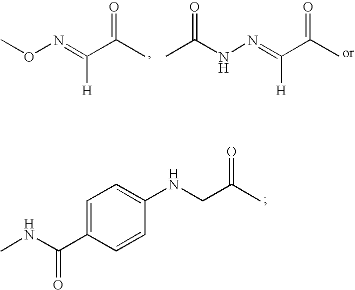
and
R is a radical containing a bulky group interfering with the binding to the compound to the prolactin receptor.

4. A compound according to claim 3 of formula (Ia) 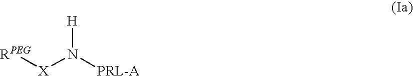
wherein
PRL-A represents a radical of a polypeptide, which polypeptide is capable of binding to the prolactin receptor;
X represents a linker selected from CH₂, 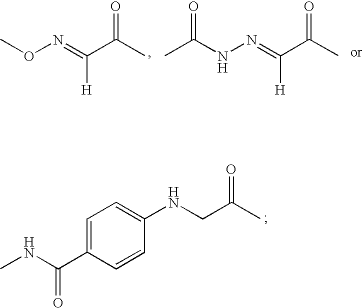
and
R^{PEG} is a polyethylene glycol containing radical.

5. A compound according to claim 3, wherein X represents a group of formula (i)

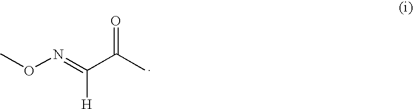

6. A process for preparing a compound of formula (Ia) as defined in claim 4, wherein X represents a group of formula 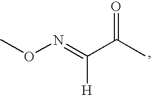
as defined in claim 4, which process comprises reaction of a compound of formula (II): 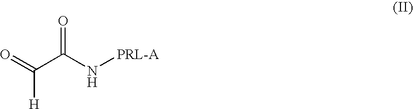
wherein PRL-A is as defined in claim 4, with a compound of formula (III): 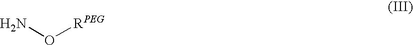
wherein R^{PEG} is as defined in claim 4.

7. A process for preparing a compound of formula (Ia) as defined in claim 4, wherein X represents CH₂, which process comprises reaction of a compound of formula (IV): 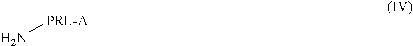
wherein PRL-A is as defined in claim 4, with a compound of formula (V): 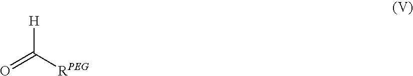
wherein R^{PEG} is as defined in claim 4.

8. A process for preparing a compound of formula (Ia) as defined in claim 4, wherein X represents 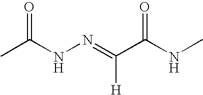
which comprises reaction of a compound of formula (II): 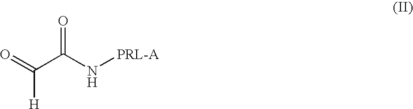
wherein PRL-A is as defined in claim 4, with a compound of formula (VII): 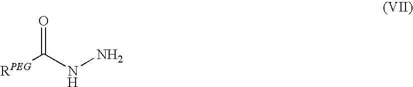
wherein R^{PEG} is as defined in claim 4.

9. A process for preparing a compound of formula (Ia) as defined in claim 4, wherein X represents 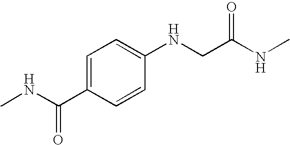
which comprises reaction of a compound of formula (II): 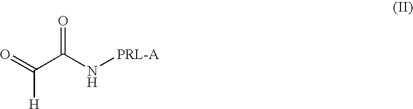
wherein PRL-A is as defined in claim 4, with a compound of formula (VIII): 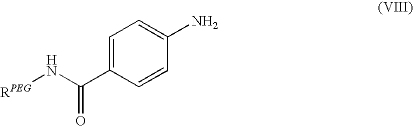
wherein R^{PEG} is as defined in claim 4.

10. A compound obtained by the process of claim 6.

11-17. (canceled)

18. A compound obtained by the process of claim 7.

19. A compound obtained by the process of claim 8.

20. A compound obtained by the process of claim 9.

21. A pharmaceutical composition comprising a compound according to claim 1.

22. A pharmaceutical composition comprising a compound according to claim 2.

23. A pharmaceutical composition comprising a compound according to claim 3.

24. A pharmaceutical composition comprising a compound according to claim 4.

25. A pharmaceutical composition comprising a compound according to claim 5.

26. A method for treating or prophylacting preventing cancer in a subject comprising administering an effective amount of the compound of claim 1 to the subject.

27. A method for treating or prophylacting preventing cancer in a subject comprising administering an effective amount of the compound of claim 2 to the subject.

28. A method for treating or prophylacting preventing cancer in a subject comprising administering an effective amount of the compound of claim 3 to the subject.

29. A method for treating or prophylacting preventing cancer in a subject comprising administering an effective amount of the compound of claim 4 to the subject.

30. A method for treating or prophylacting preventing cancer in a subject comprising administering an effective amount of the compound of claim 5 to the subject.

31. A method for treating or prophylacting preventing cancer in a subject comprising administering an effective amount of the compound of claim 10 to the subject.

32. A method for treating or prophylacting preventing cancer in a subject comprising administering an effective amount of the compound of claim 18 to the subject.

33. A method for treating or prophylacting preventing cancer in a subject comprising administering an effective amount of the compound of claim 19 to the subject.

34. A method for treating or prophylacting preventing cancer in a subject comprising administering an effective amount of the compound of claim 20 to the subject.
